(19) United States
(12) Patent Application Publication
(10) Pub. No.: US 2011/0218210 A1
(43) Pub. Date: Sep. 8, 2011

(54) COMPOUNDS FOR TREATING ABNORMAL CELLULAR PROLIFERATION

(75) Inventors: Yosef Refaeli, Denver, CO (US); Brian Curtis Turner, Denver, CO (US)

(73) Assignee: Taiga Biotechnologies, Aurora, CO (US)

(21) Appl. No.: 12/809,055

(22) PCT Filed: Nov. 3, 2008
(86) PCT No.: PCT/US08/82263

§ 371 Date: Jun. 17, 2010

Related U.S. Application Data
(60) Provisional application No. 61/050,110, filed on May 2, 2008.
(60) Provisional application No. 60/985,012, filed on Nov. 2, 2007.

(51) Int. Cl. A61K 31/519; C12Q 1/18; C07D 487/04; C12N 5/095; C07D 221/12; C07D 239/60; A61K 31/473; A61K 31/515; A61P 35/00

(52) U.S. Cl. 514/265.1; 435/32; 544/280; 435/375; 546/108; 544/305; 514/298; 514/270

(57) ABSTRACT

Provided herein are compounds, compositions and methods for treating disorders mediated by abnormal cellular proliferation and processes for identifying such compounds.

### CROSS-REFERENCE

This application claims the benefit of U.S. Provisional Application No. 60/985,012, filed Nov. 2, 2007, and U.S. Provisional Application No. 61/050,110, filed May 2, 2008, which applications are incorporated herein by reference.

### STATEMENT AS TO FEDERALLY SPONSORED RESEARCH

This invention was made with the support of the United States government under Contract number RO1 CA117802 by the National Cancer Institute (NCI) of the National Institute of Health (NIH).

### FIELD OF THE INVENTION

Described herein are compounds, compositions, methods for treating abnormal cellular proliferation, and assays and methods for discovering and developing compounds for treating abnormal cellular proliferation.

### BACKGROUND OF THE INVENTION

In certain instances, abnormal cell growth or proliferation is caused by defects or dysfunctions in cell growth control and/or regulation of apoptosis. These defects or dysfunctions can lead to abnormal cell growth and uncontrolled proliferation of cells.

Abnormal cell growth or proliferation is a characteristic of a number of disorders. Neoplasia involves a process of rapid cellular proliferation. In some instances, ncoplastic growth causes the formation of a mass of tissue (e.g., a solid neoplasm or tumor), and in others, such masses are not formed (e.g., leukemia). Proliferative disorders include cancers and other proliferative disorders. Neoplastic growth may be benign or malignant.

Proliferative disorders include, by way of non-limiting example, polyclonal proliferative diseases and/or lymphoproliferative diseases. Such diseases include, but are not limited to, lymphomas (i.e., solid neoplasms), leukemias (i.e., disseminated neoplasms), asthma, post-transplant lymphoproliferative disease (PTLD), Castleman's disease, angioimmunoblastic lymphadenopathy, X-linked lymphoproliferative disorders, Epstein Barr Virus (EBV)-associated lymphoproliferative disorder, Wiskott-Aldrich syndrome, ataxia telangiectasia, myeloproliferative disease, thrombocytosis, multiple myeloma, and a variety of autoimmune diseases (e.g., diabetes, Sjögren's syndrome, multiple sclerosis, scleroderma pigmentosa, Multiple Gammopathy of unspecified source (MGUS), Waldentröms' macroglobulinemia, myasthenia gravis), and other such maladies.

### SUMMARY OF THE INVENTION

Provided herein, in certain embodiments, are phatinaceutical composition for killing (e.g., inducing apoptosis) and/or slowing the growth of (e.g., by full or partial inhibition of proliferation and/or division) a stem cell (e.g., a cancer stem cell). Further provided herein are methods for killing (e.g., inducing apoptosis) and/or slowing the growth of (e.g., by full or partial inhibition of proliferation and/or division) a stem cell (e.g., a cancer stem cell). Further provided herein, in certain embodiments, is an assay for identifying a compound (e.g., a therapeutic agent) that kills (e.g., induces apoptosis) and/or slows the growth of (e.g., by full or partial inhibition of proliferation and/or division) a stem cell (e.g., a cancer stem cell). Further provided herein, in certain embodiments, is a method for treating a disorder (e.g., a neoplasia) characterized by the abnormal proliferation (e.g., hyperproliferation) of a cell, including a stem cell. Further provided herein, in certain embodiments, is a method for treating an autoimmune disorder.

Provided herein are compounds, pharmaceutical compositions and methods for treating proliferative disorders and for inhibiting the growth of; killing; and/or inducing apoptosis in abnormally proliferating cells. Including within the scope of such compounds, pharmaceutical compositions and methods are those in which abnormally proliferating stem cells are selectively inhibited (which includes inhibiting the proliferation of (used interchangeably herein with inhibiting the growth of), killing, and/or inducing apoptosis) relative to normally proliferating stem cells or indeed any other normally proliferating cells.

In some embodiments, provided herein is a pharmaceutical composition comprising a therapeutically effective amount a compound having the formula:

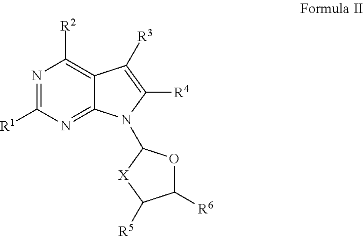

In certain embodiments, each of R¹, R², R⁵ and R⁶ is independently selected from H, OR⁷, N(R⁷)₂, N(R⁷)N(R⁷)₂, S(O)ₙR⁷, COR⁷, CON(R⁷)₂, COOR⁷, cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; X is (C(R⁸)₂)ₘ; each R⁷ and R⁸ is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; n is 0-3; m is 1-3; or a pharmaceutically acceptable salt thereof. In specific embodiments, each of R¹, R², R³, R⁴, R⁵ and R⁶ is independently selected from H, OR⁷, N(R⁷)₂, CON(R⁷)₂, COOR⁷, alkyl, heteroalkyl, and hydroxyalkyl; X is C(R⁸)₂; each R⁷ and R⁸ is independently selected from H and alkyl; or a pharmaceutically acceptable salt thereof. Tn a more specific embodiments, the compound is 4-amino-7-[(2R,3S,4S,5R)-3,4-dihydroxy-5-(hydroxymethyl)oxolan-2-yl]-6-hydrazinylpyrrolo[5,4-d]pyrimidinc-5-carboxamide or a pharmaceutically acceptable salt thereof.

In certain embodiments are compounds having the structure of Formula II, as described above or elsewhere herein.

In further or alternative embodiments, the therapeutically effective amount of a compound of Formula II is an amount sufficient to inhibit the proliferation of (used interchangeably herein with inhibit the growth of), kill, and/or induce apoptosis in cancer stem cells when the pharmaceutical composition is administered to an individual in need thereof.

In further or alternative embodiments, a pharmaceutical composition comprising a compound of Formula II, further comprises a therapeutically effective amount of an additional chemotherapeutic agent. In specific embodiments, the chemotherapeutic agents are selected from, by way of non-limiting example, alkylating agents, topoisomerase inhibitors, taxanes, cytotoxic agents, antimetabolic agents, antiangiogenesis agents, antiproliferative agents, and combinations thereof.

Provided in some embodiments herein is a method of inducing apoptosis in or inhibiting the growth of a cell comprising contacting the cell with an effective amount of a compound of Formula II. In some embodiments, the cell is a cancer stem cell. In further embodiments, the cancer stem cell is a hematological cancer stem cell. In still further embodiments, the cancer stem cell is a leukemic stem cell. In further or alternative embodiments, the cancer stem cell is present in an individual diagnosed with, is suspected of having, or is predisposed to develop cancer.

Further provided herein is a method of treating a hyperproliferative disorder by administering to an individual in need thereof a therapeutically effective amount of a compound of Formula II. In some embodiments, the hyperproliferative disease is cancer. In specific embodiments, cancer is selected from, by way of non-limiting example, a leukemia, lymphomas, other hematopoietic neoplasias, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, retinal cancer, neuronal cancer, glial malignancies, nerve-sheath tumors, and metastatic cancers thereof. In further or alternative embodiments, the cancer is, by way of non-limiting example, a hematological malignancy. In specific embodiments, the hematological malignancy is selected from B-cell Non-Hodgkin's Lymphoma (NHL), Hodgkin's Disease, B cell chronic lymphocytic leukemia/lymphoma (B-CLL), multiple myeloma and chronic myelogenous leukemia. In more specific embodiments, the B cell NHL is B cell chronic lymphocytic leukemia/lymphoma (B-CLL), Burkitt's lymphoma (BL), Follicular-like lymphoma (FLL), Diffuse large B-cell lymphoma (DLBCL), multiple myeloma, acute myeloid leukemia (AML), pre-B acute lymphocytic leukemia (ALL), pre-T acute lymphocytic leukemia (ALL), acute promyelocytic leukemia (APL), or refractory leukemia.

In certain embodiments, the hyperproliferative disease is selected from asthma, post-transplant asthma, post-transplant lymphoproliferative disease (PTLD), Castleman's disease, angioimmunoblastic lymphadenopathy, X-linked lymphoproliferative disorders, Epstein Barr Virus (EBV)-associated lymphoproliferative disorder, Wiskott-Aldrich syndrome, ataxia telangiectasia, myeloproliferative disease, thrombocytosis, multiple myeloma, an autoimmune disease, multiple gammopathy of unspecified source (MGUS), Waldentröms' macroglobulinemia, polycythemia vera (PVC), and post-transplant lymphoproliferative disease (PTLD). In specific embodiments, the autoimmune disease is selected from diabetes, aplastic anemia, Sjögren's syndrome, multiple sclerosis, vitiligo, scleroderma pigmentosa, rheumatoid arthritis, and myasthenia gravis.

Provided in certain embodiments herein is a method of treating a disorder mcdiatcd by a protooncogene (e.g., Myc), an anti-apoptosis protein (e.g., bcl-2) or a combination thereof by administering a therapeutically effective amount of a compound of Formula II.

In some embodiments, provided herein is a pharmaceutical composition comprising a therapeutically effective amount a compound having the formula:

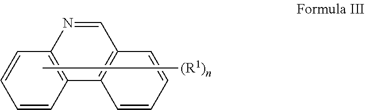

In certain embodiments, each R¹ is independently selected from H, OR², N(R²)₂, S(O)ₘR², COR², CON(R²)₂, COOR², cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; each R² is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; n is 1-6; m is 0-3; or a pharmaceutically acceptable salt thereof. In specific embodiments, n is 3. In more specific embodiments, the compound of Formula III has the structure:

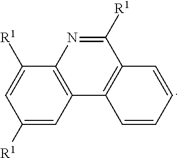

in certain embodiments are compounds having the structure of Formula III, as described above or elsewhere herein.

In more specific embodiments, the compound is 2-[(2-chloro-4-nitrophenanthridin-6-yl)amino]ethanol or a pharmaceutically acceptable salt thereof.

In certain embodiments, the therapeutically effective amount is an amount of a compound of Formula III sufficient to induce apoptosis in cancer stem cells when the pharmaceutical composition is administered to an individual in need thereof. In further or alternative embodiments, the composition comprising a compound of Formula III further comprises a therapeutically effective amount of an additional chemotherapeutic agent. In specific embodiments, the chemotherapeutic agent is selected from, by way of non-limiting example, alkylating agents, topoisomerase inhibitors, taxanes, cytotoxic agents, antimetabolic agents, antiangiogenesis agents, antiproliferative agents, and combinations thereof.

Provided in certain embodiments herein is a method of inducing apoptosis in or inhibiting the growth of a cell comprising contacting the cell with an effective amount of a compound of Formula III. In some embodiments, the cell is a cancer stem cell. In specific embodiments, the cancer stem cell is a hematological cancer stem cell. In more specific embodiments, the cancer stem cell is a leukemic stem cell. In further or alternative embodiments, the cancer stem cell is present in an individual diagnosed with, is suspected of having, or is predisposed to develop cancer.

In some embodiments, provided herein is a method of treating a hyperproliferative disorder by administering to an individual in need thereof a therapeutically effective amount of a compound of Formula III. In certain embodiments, the hyperproliferative disease is cancer. In specific embodiments, the cancer is selected from, by way of non-limiting example, a leukemia, lymphomas, other hematopoietic neoplasias, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosareomas, hemangiosarcomas, mast cell tumors, primary hepatic cancers, lung cancers, pancreatic cancers, gastrointestinal cancers, renal cell carcinomas, retinal cancer, neuronal cancer, glial malignancies, nerve-sheath tumors, and metastatic cancers thereof. In further or alternative embodiments, the cancer is, by way of non-limiting example, a hematological malignancy. In specific embodiments, the hematological malignancy is selected from, by way of non-limiting example, B-cell Non-Hodgkin's Lymphoma (NHL), Hodgkin's Disease, B cell chronic lymphocytic leukemia/lymphoma (B-CLL), multiple myeloma and chronic myelogenous leukemia. In more specific embodiments, the B cell NHL is, by way of non-limiting example, B cell chronic lymphocytic leukemia/lymphoma (B-CLL), Burkitt's lymphoma (BL), Follicular-like lymphoma (FLL), Diffuse large B-cell lymphoma (DLBCL), multiple myeloma, acute myeloid leukemia (AML), pre-B acute lymphocytic leukemia (ALL), pre-T acute lymphocytic leukemia (ALL), acute promyelocytic leukemia (APL), or refractory leukemia.

In some embodiments, the hyperproliferative disease is selected from, by way of non-limiting example, asthma, post-transplant asthma, post-transplant lymphoproliferative disease (PTLD), Castleman's disease, angioimmunoblastic lymphadenopathy, X-linked lymphoproliferative disorders, Epstein Barr Virus (EBV)-associated lymphoproliferative disorder, Wiskott-Aldrich syndrome, ataxia telangiectasia, myeloproliferative disease, thrombocytosis, multiple myeloma, an autoimmune disease, multiple gammopathy of unspecified source (MGUS), Waldentröms' macroglobulinemia, polycythemia vera (PVC), and post-transplant lymphoproliferative disease (PTLD). In specific embodiments, the autoimmune disease is selected from, by way of non-limiting example, diabetes, aplastic anemia, Sjögren's syndrome, multiple sclerosis, vitiligo, scleroderma pigmentosa, rheumatoid arthritis, and myasthenia gravis.

Provided in certain embodiments herein is a method of treating a disorder mediated by a protooncogene (e.g., Myc), an anti-apoptosis protein (e.g., bcl-2) or a combination thereof by administering a therapeutically effective amount of a compound of Formula III.

In some embodiments, provided herein is a pharmaceutical composition comprising a therapeutically effective amount a compound having the formula:

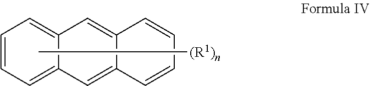

In certain embodiments, each R¹ is independently selected from H, OR², N(R²)₂, S(O)ₘR², COR², CON(R²)₂, COOR², cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; and at least one R¹ is the group:

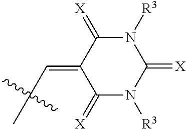

each R² is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; each X is independently selected from O, S, and NR⁴; each R³ and R⁴ is independently selected from H and alkyl; n is 1-6; m is 0-3; or a pharmaceutically acceptable salt thereof. In specific embodiments, n is 1, 2 or 3. In further or alternative embodiments, the compound of Formula IV has a structure of Formula V:

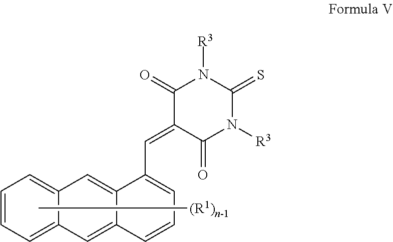

In more specific embodiments, the compound is 5-(anthracen-1-ylmethylidene)-2-sulfanylidene-1,3diazinane-4,6-dione or a pharmaceutically acceptable salt thereof.

In certain embodiments are compounds having the structure of Formula IV or Formula V, as described above or elsewhere herein.

In some embodiments, the therapeutically effective amount is an amount of a compound of Formula IV sufficient to induce apoptosis in cancer stem cells when the pharmaceutical composition is administered to an individual in need thereof. In further or alternative embodiments, the composition comprising a compound of Formula IV further comprises a therapeutically effective amount of an additional chemotherapeutic agent. In specific embodiments, the chemotherapeutic agent is selected from, by way of non-limiting example, alkylating agents, topoisomerase inhibitors, taxanes, cytotoxic agents, antimetabolic agents, antiangiogenesis agents, antiproliferative agents, and combinations thereof.

Provided in certain embodiments herein is a method of inducing apoptosis in or inhibiting the growth of a cell comprising contacting the cell with an effective amount of a compound of Formula IV or V. In specific embodiments, the cell is a cancer stem cell. In more specific embodiments, the cancer stem cell is a hematological cancer stem cell. In still more specific embodiments, the cancer stem cell is a leukemic stem cell. In further or alternative embodiments, the cancer stem cell is present in an individual diagnosed with, is suspected of having, or is predisposed to develop cancer.

Provided in some embodiments herein is a method of treating a hyperproliferative disorder by administering to an individual in need thereof a therapeutically effective amount of a compound of Formula IV or V. In specific embodiments, the hyperproliferative disease is cancer. In more specific embodiments, the cancer is selected from, by way of non-limiting example, a leukemia, lymphomas, other hematopoietic neoplasias, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcoma, hemangiosarcomas, mast cell tumors, primary hepatic cancers, lung cancers, pancreatic cancers, gastrointestinal cancers, renal cell carcinomas, retinal cancer, neuronal cancer, glial malignancies, nerve-sheath tumors, and metastatic cancers thereof. In further or alternative embodiments, the cancer is, by way of non-limiting example, a hematological malignancy. In specific embodiments, the hematological malignancy is selected from, by way of non-limiting example, B-cell Non-Hodgkin's Lymphoma (NHL), Hodgkin's Disease, B cell chronic lymphocytic leukemia/lymphoma (B-CLL), multiple myeloma and chronic myelogenous leukemia. In more specific embodiments, the B cell NHL is, by way of non-limiting example, B cell chronic lymphocytic leukemia/lymphoma (B-CLL), Burkitt's lymphoma (BL), Follicular-like lymphoma (FLL), Diffuse large B-cell lymphoma (DLBCL), multiple myeloma, acute myeloid leukemia (AML), pre-B acute lymphocytic leukemia (ALL), pre-T acute lymphocytic leukemia (ALL), acute promyelocytic leukemia (APL), or refractory leukemia.

In some embodiments, the hyperproliferative disease is selected from, by way of non-limiting example, asthma, post-transplant asthma, post-transplant lymphoproliferative disease (PTLD), Castleman's disease, angioimmunoblastic lymphadenopathy, X-linked lymphoproliferative disorders, Epstein Barr Virus (EBV)-associated lymphoproliferative disorder, Wiskott-Aldrich syndrome, ataxia telangiectasia, myeloproliferative disease, thrombocytosis, multiple myeloma, an autoimmune disease, multiple gammopathy of unspecified source (MGUS), Waldentröms' macroglobulinernia, polycythemia vera (PVC), and post-transplant lymphoproliferative disease (PTLD). In specific embodiments, the autoimmune disease is selected from, by way of non-limiting example, diabetes, aplastic anemia, Sjögren's syndrome, multiple sclerosis, vitiligo, scleroderma pigmcntosa, rheumatoid arthritis, and myasthenia gravis.

Provided in certain embodiments herein is a method of treating a disorder mediated by a protooncogene (e.g., Myc), an anti-apoptosis protein (e.g., bcl-2) or a combination thereof by administering a therapeutically effective amount of a compound of Formula IV or V.

In some embodiments, provided herein is a composition comprising a therapeutically effective amount of a compound in an amount sufficient to selectively induce apoptosis in cancer stem cells relative to non-cancer stem cells when the composition is administered to an individual having both cancer stem cells and non-cancer stem cells. In specific embodiments, the stem cells are hematopoietic stem cells.

Provided in certain embodiments herein is a process for identifying a therapeutic agent that selectively inhibit the growth of, induce apoptosis in, or a combination thereof in cancer stem cells by:
a. presenting a conditionally immortalized hematopoietic stem cell and a hematologic cancer stem cell;
b. contacting the conditionally immortalized hematopoietic stem cell and the hematologic cancer stem cell with a candidate compound;
c. detecting or measuring the impact of the candidate compound on viability of the conditionally immortalized hematopoietic stem cell and the hematologic cancer stem cell;
d. comparing the impact of the candidate compound on viability of the conditionally immortalized hematopoietic stem cell to the impact of the candidate compound on viability of the hematologic cancer stem cell.

In some embodiments, the conditionally immortalized hematopoietic stem cell comprises recombinant MYC-ER and bcl-2 polypeptides. In further or alternative embodiments, the hematologic cancer stem cell is a leukemic stem cell. In further or alternative embodiments, the MYC-ER is selected from Tat-MYC-ER and Vpr-MYC ER. In further or alternative embodiments, the bcl-2 is Tat-Bcl-2 and Vpr-Bcl-2. In further or alternative embodiments, detecting or measuring the impact of the candidate compound on viability of the conditionally immortalized hematopoietic stem cell and the hematologic cancer stem cell is achieved by 7AAD staining, a GFP viability assay, or a combination thereof. In some embodiments, provided herein is a therapeutic agent identified by a process for identifying a therapeutic agent that selectively inhibit the growth of, induce apoptosis in, or a combination thereof in cancer stem cells.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates that the compounds disclosed herein reduce viability of leukemic stem cells but not normal stein cells. Normal murine hematopoetic stem cell line (ctlt-HSC cell line) (BL6 BM) or leukemic stem cell line (ABM42C31) are incubated with serial two-fold dilutions of compounds described herein for 24 h and assayed for viability by MTS assay. Results are representative of three independent experiments. Error bars represent (+/−) the Standard Deviation of measurements from triplicate wells per condition. FIG. 1A illustrates data for the compound 2-[(2-chloro-4-nitrophenanthridin-6-yl)amino]ethanol; FIG. 1B illustrates data for the compound 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide; FIG. 1C illustrates data for the compound 5-(anthracen-1-ylmethylidene)-2-sulfanylidene-1,3diazinane-4,6-dione.

FIG. 2 illustrates that compounds disclosed herein reduce viability of leukemic stem cells via apoptosis. Leukemic stem cells (FIG. 2a) or normal murine hematopoetic stem cells (FIG. 2b) are incubated with 10 uM of the compounds for 24 h and assayed for apoptosis by retention of 4 μM 7-aminoactinomycin-D (7AAD). FIGS. 2a-α and 2b-α illustrates data for the compound 5-(anthracen-1-ylmethylidene)-2-sulfanylidene-1,3diazinane-4,6-dione; FIG. 2a-β and 2b-β illustrates data for the compound 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide; FIGS. 2a-γ and 2b-γ illustrates data for the compound 2-[(2-chloro-4-nitrophenanthridin-6-yl)amino]ethanol.

FIG. 3 illustrates that compounds disclosed herein reduce viability of human leukemia cell lines but not normal stem cell lines. Normal human hematopoetic stem cell line (FCB61107) or a leukemic cell line (U937) are incubated with serial two-fold dilutions of compounds disclosed herein for 24 h and assayed for viability by MTS assay. Results are representative of three independent experiments. Error bars represent (+/−) the Standard Deviation of measurements from triplicate wells per condition. FIG. 3 A illustrates data for the compound 2-[2-chloro-4-nitrophenanthridin-6-yl)amino]ethanol; FIG. 3 B illustrates data for the compound 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide; FIG. 3 C illustrates data for the compound 5-(anthracen-1-ylmethylidene)-2-sulfanylidene-1,3diazinane-4,6-dione.

FIG. 4 illustrates that compounds described herein do not reduce viability of primary unmanipulated fetal cord blood cells. Normal human fetal cord blood stem cells are incubated with serial two-fold dilutions of compounds described herein for 24 h and assayed for viability by MTS assay. Results are representative of three independent experiments. Error bars represent (+/−) the Standard Deviation of measurements from triplicate wells per condition. FIG. 4 A illustrates data for the compound 2-[(2-chloro-4-nitrophenanthridin-6-yl)amino]ethanol; FIG. 4 B illustrates data for the compound 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide; FIG. 4 C illustrates data for the compound 5-(anthracen-1-ylmethylidene)-2-sulfanylidene-1,3diazinane-4,6-dione.

FIG. 5. Kinetics of attrition of mice during treatment with compounds disclosed herein. Cohorts of tumor-bearing mice are treated with one of the compounds by administration of seven daily doses of drug, intravenously (250 ul of [10 uM] soln). The graph represents the rates of mortality of the mice in the treatment cohorts during treatment. We noted that untreated tumor-bearing mice die at a much higher rate than the treated mice. In addition, the life-span of a tumor bearing mouse is nearly doubled following treatment with one of the compounds. We also noted that 100× the dose used in this study had no mortality in non-tumor bearing mice. The graph is representative of three independent experiments. Line A illustrates data for the compound 2-[(2-chloro-4-nitrophenanthridin-6-yl)amino]ethanol; Line B illustrates data for the compound 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide; Line C illustrates data for the compound 5-(anthracen-1-ylmethylidene)-2-sulfanylidene-1,3diazinane-4,6-dione.

FIG. 6 illustrates that compounds of Formulas I-V kill leukemic stem cells but spare normal hematopoietic stem cells. FIG. 6A illustrates the effects of 2-[(2-chloro-4-nitrophenanthridin-6-yl)amino]ethanol on leukemic stem cells (square) and normal hematopoietic stem cells (circle). FIG. 6B illustrates the effects of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide on leukemic stem cells (square) and normal hematopoietic stem cells (circle). FIG. 6C illustrates the effects of 5-(anthracen-1-ylmethylidene)-2-sulfanylidene-1,3diazinane-4,6-dione on leukemic stem cells (square) and normal hematopoietic stem cells (circle). FIG. 6D illustrates that both on leukemic stem cells (square) and normal hematopoietic stem cells (circle) are killed by methotrexate.

FIG. 7 illustrates the effects of 2-[(2-chloro-4-nitrophenanthridin-6-yl)amino]ethanol on two human leukemic stem cell lines (HL-60 and U937) and normal hematopoietic stem cells (FCB cell line and Primary FCB). The results were obtained in two seperate experiments. Further, the results are representative of all three compounds.

FIG. 8 illustrates the dose responses to compounds of Formula I-V. FIG. 8a illustrates the effects of 2-[(2-chloro-4-nitrophenanthridin-6-yl)amino]ethanol on DBL cells. FIG. 8b illustrates the effects of 2-[(2-chloro-4-nitrophenanthridin-6-yl)amino]ethanol on TBL cells. FIG. 8c illustrates the effects of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide on DBL cells. FIG. 8d illustrates the effects of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide on TBL cells. FIG. 8e illustrates the effects of 5-(anthracen-1-ylmethylidene)-2-sulfanylidene-1,3diazinane-4,6-dione on DBL cells. FIG. 8f illustrates the effects of 5-(anthracen-1-ylmethylidene)-2-sulfanylidene-1,3diazinane-4,6-dione on TBL cells.

FIG. 9 illustrates the Rapid Therapeutic Assessment protocol.

FIG. 10 illustrates the tumor counts in lymph nodes 3 days after initial treatment in mouse models of Diffuse large B-cell lymphoma (DLBCL). The first bar illustrates the counts in mice that received no treatment. The second bar illustrates tumor counts in mice receiving treatment with 5-(anthracen-1-ylmethylidene)-2-sulfanylidene-1,3diazinane-4,6-dione. The third bar illustrates the tumor counts in mice receiving treatment with 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide. The final bar illustrates the tumor counts in mice receiving treatment with 2-[(2-chloro-4-nitrophenanthridin-6-yl)amino]ethanol.

FIG. 11 illustrates the tumor counts in spleens 3 days after initial treatment in mouse models of Diffuse large B-cell lymphoma (DLBCL). The first bar illustrates the counts in mice that received no treatment. The bar A illustrates tumor counts in mice receiving treatment with 5-(anthracen-1-ylmethylidene)-2-sulfanylidene-1,3diazinane-4,6-dione. The bar B illustrates the tumor counts in mice receiving treatment with 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide. The bar C illustrates the tumor counts in mice receiving treatment with 2-[(2-chloro-4-nitrophenanthridin-6-yl)amino]ethanol.

FIG. 12 illustrates the effectiveness of the compounds of Formulas I-IV in a mouse model of Diffuse large B-cell lymphoma (DLBCL) as a plot of percent survival versus number of days post-therapeutic assessment. This figure is representative of two independent experiments. The first line illustrates the survival of mice that received no therapeutic assessment and no treatment. The second line illustrates the survival of mice receiving therapeutic assessment and no treatment. The line A illustrates the survival of mice receiving therapeutic assessment and treatment with 2-[(2-chloro-4-nitrophenanthridin-6-yl)amino]ethanol. The line B illustrates the survival of mice receiving therapeutic assessment and treatment with 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide. The line C illustrates the survival of mice receiving therapeutic assessment and treatment with 5-(anthracen-1-ylmethylidene)-2-sulfanylidene-1,3diazinane-4,6-dione.

FIG. 13 illustrates the effectiveness of the compounds of Formulas I-IV in a mouse model of acute myeloid leukemia as a plot of percent survival versus number of days post-therapeutic assessment. This figure is representative of three independent experiments. The first line illustrates the survival of mice receiving therapeutic assessment and no treatment. The line A illustrates the survival of mice receiving therapeutic assessment and treatment with 2-[(2-chloro-4-nitrophenanthridin-6-yl)amino]ethanol. The line B illustrates the survival of mice receiving therapeutic assessment and treatment with 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide. The line C illustrates the survival of mice receiving therapeutic assessment and treatment with 5-(anthracen-1-ylmethylidene)-2-sulfanylidene-1,3diazinane-4,6-dione. The fifth line illustrates the survival of mice that received no therapeutic assessment and no treatment.

FIG. 14 illustrates the effectiveness of the compounds of Formulas I-IV in a mouse model of B cell chronic lymphocytic leukemia/lymphoma (B-CLL) as a plot of percent survival versus number of days post-therapeutic assessment. The first line illustrates the survival of mice that received no therapeutic assessment and no treatment. The second line illustrates the survival of mice receiving therapeutic assessment and no treatment. The line A illustrates the survival of mice receiving therapeutic assessment and treatment with 2-[(2-chloro-4-nitrophenanthridin-6-yl)amino]ethanol. The line B illustrates the survival of mice receiving therapeutic assessment and treatment with 5-(anthracen-1-ylmethylidene)-2-sulfanylidene-1,3diazinane-4,6-dione. The line C illustrates the survival of mice receiving therapeutic assessment and treatment with 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide.

FIG. 15 illustrates the differential activity of the compounds of Formulas I-IV on human multiple myeloma cell lines LP-1 and OPM-2. The darkened square of FIG. 15a illustrates the effect of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide on the LP-1 line. The darkened triangle of FIG. 15a illustrates the effect of 7-[3,4-dihydroxy-5-(hydroxymethyl)oxolan-2-yl]-N′-hydroxy-4-(hydroxyamino)pyrrolo[5,4-d]pyrimidine-5-carboximidamide chloride on the LP-1 line. The clear square of FIG. 15a illustrates the effects of 5-(anthracen-1-ylmethylidene)-2-sulfanylidene-1,3diazinane-4,6-dione on the LP-1 line. The clear triangle of FIG. 15a illustrates the effects of 2-[(2-chloro-4-nitrophenanthridin-6-yl)amino]ethanol on the LP-1 line. The darkened square of FIG. 15b illustrates the effect of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide on the OPM-2 line. The darkened triangle of FIG. 15b illustrates the effect of 7-[3,4-dihydroxy-5-(hydroxymethyl)oxolan-2-yl]-N′-hydroxy-4-(hydroxyamino)pyrrolo[5,4-d]pyrimidine-5-carboximidamide chloride on the OPM-2 line. The clear square of FIG. 15b illustrates the effects of 5-(anthracen-1-ylmethylidene)-2-sulfanylidene-1,3diazinane-4,6-dione on the OPM-2 line. The clear triangle of FIG. 15b illustrates the effects of 2-[(2-chloro-4-nitrophenanthridin-6-yl)amino]ethanol on the OPM-2 line.

FIG. 16 illustrates that the compounds of Formulas I-V are less toxic to normal mice than methotrexate (MTX) as measured by the weight of the mice post administration. The darkened squares illustrate the weight of the mice not administed any compounds. The darkend triangles illustrate the weight of mice administered methotrexate. The clear square illustrate the weight of mice administred 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide. The clear triangles illustrate the weight of mice administered 5-(anthracen-1-ylmethylidene)-2-sulfanylidene-1,3diazinane-4,6-dione. The darkened diamonds illustrate the weight of mice administered 2-[(2-chloro-4-nitrophenanthridin-6-yl)amino]ethanol.

FIG. 17 illustrates that treatment of mice with 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide results in less bone marrow toxicity as compared to mice treated with methotrexate (MTX). The results are representative of the results obtained when administering 5-(anthracen-1-ylmethylidene)-2-sulfanylidene-1,3diazinane-4,6-dione or 2-[(2-chloro-4-nitrophenanthridin-6-yl)amino]ethanol.

### DETAILED DESCRIPTION OF THE INVENTION

Provided in certain embodiments herein are compounds, compositions and methods for treating a proliferative disorder. In certain instances, proliferative disorders are characterized by abnormal cellular proliferation and/or abnormal cellular apoptosis. Typically, hyperproliferative disorders are characterized by increased cellular proliferation and/or decreased cellular apoptosis. In some embodiments, provided herein is a method of treating a proliferative disorder by administering a therapeutically effective amount of any compound described herein to an individual in need thereof. In some embodiments, provided herein is a method of treating a proliferative disorder by administering any pharmaceutical composition described herein to an individual in need thereof. In some embodiments, the proliferative disorder is a hematological proliferative disorder. In certain embodiments, hematological proliferative disorder is selected from, by way of non-limiting example, a lymphoproliferative disorder and a myeloproliferative disorder. In some embodiments, the proliferative disorder is cancer. In certain embodiments, the hematological proliferative disorder is a hematological cancer. In certain embodiments, the proliferative disorder is an autoimmune disease.

In certain instances, the proliferative disorder is mediated by the unrestricted growth of cells. In certain instances, the proliferative disorder is mediated by the unrestricted growth of stem cells (e.g., human stem cells). In some embodiments, administration of a compound or composition described herein effectively kills, induces apoptosis in and/or inhibits proliferation of stem cells characterized by unrestricted cell growth, while sparing normal stem cells (i.e., stem cells characterized by normal cell growth, e.g., wild type and/or conditionally immortalized stem cells under non-immortalizing conditions). In certain embodiments, the stem cells characterized by unrestricted cell growth are cancer stem cells, such as, by way of non-limiting example, hematological cancer stein cells (e.g., leukemic stem cells). In some instances, conventional cancer therapy targets cancer cells that do not have stem cell characteristics (e.g., the cells that comprise the bulk of a tumor mass) without affecting the cancer stem cells. Thus, in certain instances, the conventional treatment of cancer (e.g., leukemia) allows the cancer to recur following, e.g., relapse or remission of the cancer. Accordingly, in some embodiments, a method of treating cancer described herein includes a prophylactic treatment of cancer following conventional cancer therapy. Furthermore, in some embodiments, provided herein is a method of treating a cancer with a compound described herein in combination with a therapy for treating or targeting cancer cells that do not have stem cell characteristics (e.g., the cells that comprise the bulk of a tumor mass). In certain embodiments, a method described herein includes a method of treating a proliferative disorder (e.g., cancer) that is refractory to a conventional cancer therapy. In some embodiments, the compounds described herein target the stem cells characterized by unrestricted cell growth, while sparing normal stem cells. In certain instances, this allows normal stem cells to improve an individual's ability to withstand cancer therapy (e.g., the side effects caused by conventional chemotherapeutic approaches that can leave the hematopoietic system and other body systems weak). In certain instances, a cancer stem cell is the initiating population from which the bulk of the cancer, tumor or leukemia emerges. In some instances, the nature and characteristics of the cancer stem cell is different from the bulk of the cancer, tumor or leukemia it gives rise to. In certain instances, leukemic stem cells resemble a normal hematopoietic stem cell.

### Compounds

In certain embodiments, therapeutic compounds disclosed herein include compounds that target (e.g., selectively kill, induce apoptosis in and/or inhibit the growth of) cells characterized by unrestricted cell growth, while completely or partially sparing normal (or wild type) cells (e.g., of the same lineage). In certain embodiments, therapeutic compounds disclosed herein include compounds that target (i.e., selectively kill, induce apoptosis in and/or inhibit) stem cells characterized by unrestricted cell growth, while completely or partially sparing normal (or wild type) stem cells. In some embodiments, the stem cell characterized by unrestricted growth and the normal stem cell is a hematopoietic stem cell. In some embodiments, the therapeutic compound is a compound of any of Formulas I-V.

In some embodiments, the therapeutic compounds disclosed herein are compounds that inhibit or disrupt the metabolism of glucose (e.g., glucose catabolism). In specific embodiments, the therapeutic compound is a compound of any of Formulas I-V. In some embodiments, the therapeutic compound is a compound of either of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide; and 5-(anthracen-1-ylmethylidene)-2-sulfanylidene-1,3diazinane-4,6-dione.

In certain embodiments, the therapeutic compound disclosed herein is a compound of Formula I:

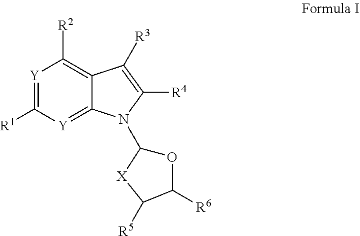

In certain embodiments, each Y is independently selected from N and CR′. In some embodiments, each of R′, R¹, R², R³, R⁴, R⁵ and R⁶ is independently selected from H, OR⁷, N(R⁷)₂, N(R⁷)N(R⁷)₂, S(O)ₙR⁷, COR⁷, CON(R⁷)₂, COOR⁷, cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl. In certain embodiments, R¹ and an R′, or R² and R′, or R² and R³, or R³ and R⁴ are taken together to form (C(R″)₂)ₚ, wherein p is 1-4, and wherein one or more of (C(R″)₂)ₚ is optionally substituted with NR″ or O. In certain embodiments, R″ is selected from H, OR⁷, N(R⁷)₂, S(O)ₙR⁷, COR⁷, CON(R⁷)₂, COOR⁷, cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl, or one or more pair of adjacent R″ groups, taken together, form a double bond. In some embodiments, X is (C(R⁸)₂)ₘ. In certain embodiments, each R⁷ and R⁸ is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl. In some embodiments, n is 0-3 and m is 1-3. In some embodiments, the therapeutic compound is a pharmaceutically acceptable salt, tautomer, prodrug, metabolite, solvate, stereoisomer, or polymorph of a compound of Formula I.

In certain embodiments are compounds having the structure of Formula I, as described above or elsewhere herein.

In some embodiments, the therapeutic compound disclosed herein is a compound of Formula II:

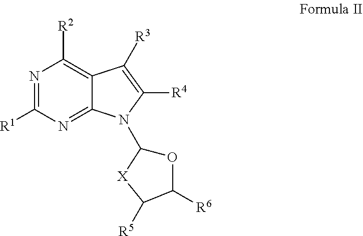

In certain embodiments, the terms of Formula II are as set forth in Formula I. In specific embodiments, each of R¹, R², R³, R⁴, R⁵ and R⁶ is independently selected from H, OR⁷, N(R⁷)₂, N(R⁷)N(R⁷)₂, S(O)ₙR⁷, COR⁷, CON(R⁷)₂, COOR⁷, cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; X is (C(R⁸)₂)ₘ; each R⁷ and R⁸ is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl; n is 0-3; and m is 1-3. In some embodiments, the therapeutic compound is a pharmaceutically acceptable salt, tautomer, prodrug, metabolite, solvate, stereoisomer, or polymorph of a compound of Formula II.

In specific embodiments, R¹ is H or alkyl; R² is H or N(R⁷)₂; R³ is H or CON(R⁷)₂; R⁴ is H or N(R⁷)N(R⁷)₂; R⁵ is H or OR⁷; R⁶ is H or OR⁷; m is 1; and R⁸ is H or hydroxyalkyl. In more specific embodiments, the therapeutic compound is 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide or pharmaceutically acceptable salt, tautomer, prodrug, metabolite, solvate, stereoisomer, or polymorph thereof.

In certain embodiments, the therapeutic compound disclosed herein is a compound of Formula III:

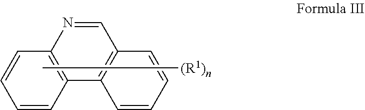

In some embodiments, each R¹ is independently selected from OR², N(R²)₂, S(O)ₘR², COR², CON(R²)₂, COOR², cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl. In certain embodiments, each R² is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted awl, substituted or unsubstituted heteroaryl. In some embodiments, n is 1-6 and m is 0-2. In certain embodiments, n is selected from 1, 2, 3, 4, 5 and 6. In some embodiments, the therapeutic compound is a pharmaceutically acceptable salt, tautomer, prodrug, metabolite, solvate, stereoisomer, or polymorph of a compound of Formula III.

In specific embodiments, each R¹ is individually selected from N(R²)₂, nitro and halo. In more specific embodiments, n is 3-4. In still more specific embodiments, n is 3. In yet a more specific embodiments, the therapeutic compound is 2-[(2-chloro-4-nitrophenanthridin-6-yl)amino]ethanol or pharmaceutically acceptable salt, tautomer, prodrug, metabolite, solvate, stereoisomer, or polymorph thereof.

In certain embodiments, the therapeutic compound disclosed herein is a compound of Formula IV:

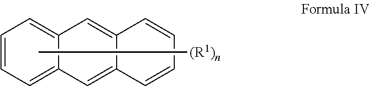

In some embodiments, each R¹ is independently selected from OR², N(R²)₂, S(O)ₘR², COR², CON(R²)₂, COOR², cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, substituted or unsubstituted heteroaryl, provided that at least one R¹ is the group:

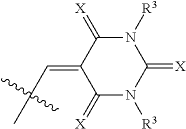

In certain embodiments, each R² is independently selected from H, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, and substituted or unsubstituted heteroaryl. In some embodiments, each X is independently selected from O, S, and NR⁴. In certain embodiments, each R³ and R⁴ is independently selected from H and alkyl. In some embodiments, n is 1-6 and m is 0-3. In certain embodiments, n is selected from 1, 2, 3, 4, 5 and 6. In some embodiments, the therapeutic compound is a pharmaceutically acceptable salt, tautomer, prodrug, metabolite, solvate, stereoisomer, or polymorph of a compound of Formula IV.

In certain embodiments, the therapeutic compound disclosed herein is a compound of Formula V:

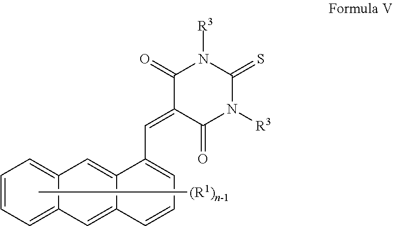

In some embodiments, R¹, R³ and n are as described above for Formula V. In certain embodiments, each R¹ is independently selected from OR², N(R²)₂, S(O)ₘR², COR², CON(R²)₂, COOR², cyano, nitro, halo, substituted or unsubstituted alkyl, substituted or unsubstituted heteroalkyl, substituted or unsubstituted hydroxyalkyl, substituted or unsubstituted cycloalkyl, substituted or unsubstituted heterocycloalkyl, substituted or unsubstituted aryl, and substituted or unsubstituted heteroaryl, wherein R² and m are as described for Formula IV. In some embodiments, the therapeutic compound is a pharmaceutically acceptable salt, tautomer, prodrug, metabolite, solvate, stereoisomer, or polymorph of a compound of Formula V.

In specific embodiments, n is 1-3 and each R¹ is independently selected from OR², N(R²)₂, COOR², cyano, nitro, halo, alkyl, and heteroalkyl. In more specific embodiments, the therapeutic compound is 5-(anthracen-1-ylmethylidene)-2-sulfanylidene-1,3diazinane-4,6-dione or pharmaceutically acceptable salt, tautomer, prodrug, metabolite, solvate, stereoisomer, or polymorph thereof.

In certain embodiments, therapeutic compounds described herein have one or more chiral centers. As such, all stereoisomers are envisioned herein. In various embodiments, therapeutic compounds described herein are present in optically active or racemic forms. It is to be understood that the compounds of the present invention encompasses racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, therapeutic compounds described herein contains one or more chiral centers. These compounds are prepared by any means, including entioselective synthesis and/or separation of a mixture of enantiomers and/or diastereomers. Resolution of therapeutic compounds and isomers thereof is achieved by any means including, by way of non-limiting example, chemical processes, enzymatic processes, fractional crystallization, distillation, chromatography, and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as disclosed herein are modified by the use of appropriate reagents and conditions, for the introduction of the various moieties found in the formulae as provided herein. As a guide the following synthetic methods are utilized.

Compounds described herein are synthesized starting from compounds that are available from commercial sources or that are prepared using procedures outlined herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linakges. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Aziridines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in then presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

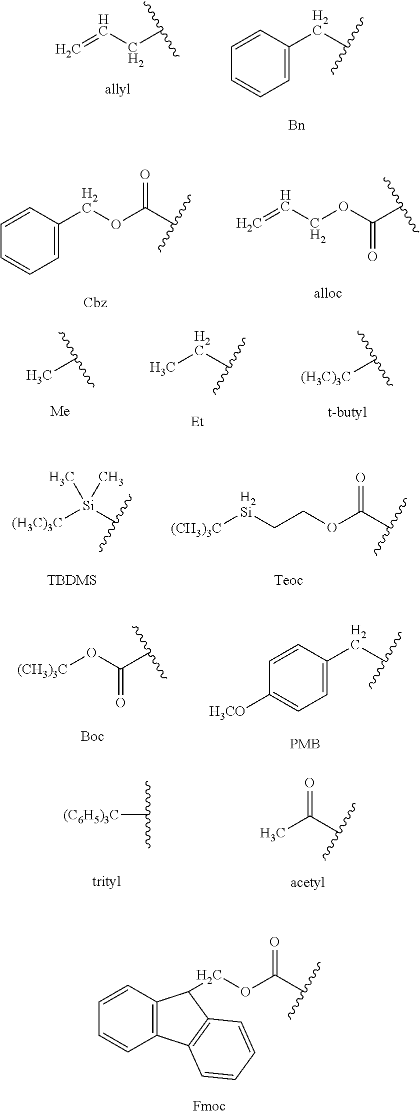

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

Provided in some embodiments herein are compounds, compositions and methods for killing, inducing apoptosis in or inhibiting the proliferation of a cell. In certain embodiments, the cell is a cell with abnormal proliferation (e.g., compared to a wild type cell of the same lineage). In some embodiments, the cell with abnormal proliferation is an abnormal stem cell or an abnormal progenitor cell. In some embodiments, the cell that is characterized by abnormal proliferation is an abnormal hematopoietic stem cell or an abnormal hematopoietic progenitor. In certain embodiments, the cell that is characterized by abnormal proliferation is a cancer stem cell (e.g., a leukemic stem cell). In some embodiments, the cell that is characterized by abnormal proliferation is a cell (e.g., an abnormal hematopoietic stem cell) that relics for energy on and/or possesses at least partially deregulated glucose metabolism. In specific embodiments, the deregulated glucose metabolism of the cell is caused by the overexpression of an oncoprotein (e.g., a Myc oncoprotein). In some embodiments, the cell that is characterized by abnormal proliferation is a cell (e.g., an abnormal hematopoietic stem cell) with increased glucose metabolism (e.g., compared to a wild type cell of the same lineage). In certain embodiments, the abnormal proliferation of a cell described herein is caused, at least in part, by a loss of regulation of or an unregulated protooncogene or oncogene; by the overexpression of an oncoprotein (as used herein, oncoprotein includes protooncoprotein); by the overexpression of an apoptosis inhibiting polypeptide; loss of a tumor suppressor gene; generation of a fusion protein between two proto-oncogenes; following a chromosomal translocation; dysregulation of cytokine/growth factor receptors by auto-heteromerization following a mutation; and the like; or a combination thereof.

### GENERAL DEFINITIONS

The term “subject,” “individual,” or “patient” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human. None of the terms require or are limited to situation characterized by the supervision (e.g constatnt or intermittent) of a health care worker (e.g. a doctor, a registered nurse, a nurse practicioner, a physican's assistant, an orderly, or a hospice worker).

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to an individual simultaneously in the form of a single entity or dosage. The term “non-fixed combination” means that at least one of the agents described herein, and at least one co-agent, are administered to an individual as separate entities either simultaneously, concurrently or sequentially with variable intervening time limits, wherein such administration provides effective levels of the two or more agents in the body of the individual. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single individual, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

As used herein, the term “stem cell” refers to any cell characterized by (1) the ability to undergo mitotic division and (2) differentiate into one or more types of cell. “Stem cell” includes any cell that is totipotent (i.e., a cell that can differentiate into any cell), pluripotent (i.e., a cell that has the ability to differentiate into endoderm, mesoderm or ectoderm; e.g., an embryonic stem cell), multipotent (i.e., a cell that can differentiate into several types of cells but not all cells; e.g., hematopoietic cell), oligopotent (i.e., a cell capable of generating a few cell types within a particular tissue e.g., vascular stem cells), or unipotent (a cell that has the capacity to differentiate into only one type of cell). “Stem cells” include progenitor cells.

As used herein, the term “cancer stem cell” includes any cell characterized by (1) the ability to undergo mitotic division and (2) differentiate into one or more types of cell found in a neoplasm. “Cancer stem cells” include any cell that is totipotent, pluripotent, multipotent, oligopotent, or unipotent. “Cancer stem cells” include progenitor cells.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, a agent of this invention or a pharmaceutically active metabolite or residue thereof. Particularly favored prodrugs are those that increase the bioavailability of the agents of this invention when such agents are administered to an individual (e.g., by allowing an orally administered agent to be more readily absorbed into blood) or which enhance delivery of the parent agent to a biological compartment (e.g., the brain or lymphatic system). In various embodiments, pharmaceutically acceptalbe salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

Glucose metabolism includes, by way of non-limiting example, catabolism of glucose, glycolysis, glycogen synthesis and the like. Glycolysis/glucose metabolism pathways include those mediated but GLUT1, hexokinase, GSK3β, Akt and/or its downstream pathway, and the like.

The terms, “abnormally proliferating cell”, “cell with abnormal proliferation”, “cell characterized by abnormal proliferation” and similar terms are used interchangeably herein and refer to a cell that abnormally proliferates compared to a normal or wild type cell of the same lineage and/or a conditionally immortalized cell of the same lineage under non-immortalizing conditions or wherein the conditional immortalization is deactivated.

As used herein, protooncogene refers to a nucleic acid that comprises an open reading frame for a protooncoprotein or an oncoprotein. In certain instances, the nucleic acid further comprises, e.g., an inducible promoter (e.g., one responsive to tetracycline or an analog thereof), a constitutively active promoter, a cell or tissue specific promoter, a long terminal repeat (LTR), an internal ribosome entry site (IRES), and/or recombinase target cites (Cre, Flp and the like). In certain instances, the protooncogene refers to a nucleic acid that encodes a fusion polypeptide comprising a protooncoprotein or oncoprotein, linked N-terminally or carboxy terminally, and a receptor (e.g., an estrogen receptor (ER)) that conditionally activates the protooncoprotein or oncoprotein when modulated (e.g., bound with a ligand, agonized or antagonized).

As used herein, a gene that encodes a polypeptide that inhibits apoptosis of the cell refers to a nucleic acid that comprises an open reading frame for a polypeptide that inhibits apoptosis of the cell. In certain intances, the nucleic acid further comprises, e.g., an inducible promoter (e.g., one responsive to tetracycline or an analog thereof), a constitutively active promoter, a cell or tissue specific promoter, a long terminal repeat (LTR), an internal ribosome entry site (IRES), and/or recombinase target cites (Cre, Flp and the like).

The term “optionally substituted” or “substituted” means that the referenced group substituted with one or more additional group(s). In certain embodiments, the one or more additional group(s) are individually and independently selected from alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O-group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

The term “carbocyclic” or “carbocycle” refers to a ring wherein each of the atoms forming the ring is a carbon atom. Carbocycles includes aryl and cycloalkyl groups. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles disclosed herein are optionally substituted.

As used herein, the term “aryl” refers to an aromatic ring wherein each of the atoms forming the ring is a carbon atom. Aryl rings disclosed herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms funning the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

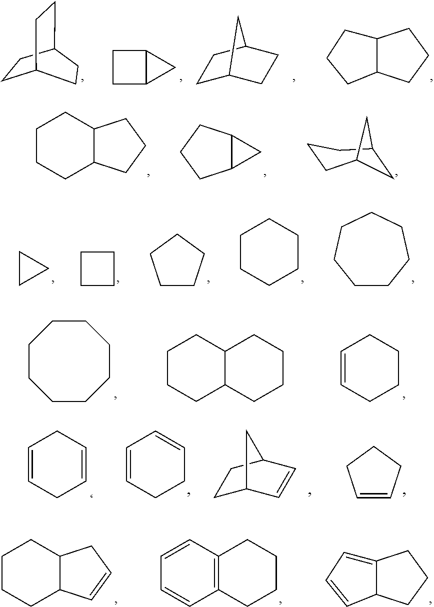

and the like. Monocyclic cycloalkyls include, but are not limited to, cyclopropyl, cyclobutyl, cyclopentyl, cyclohexyl, cycloheptyl, and cyclooctyl.

The term “heterocycle” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Illustrative examples of heteroaryl groups include the following moieties:

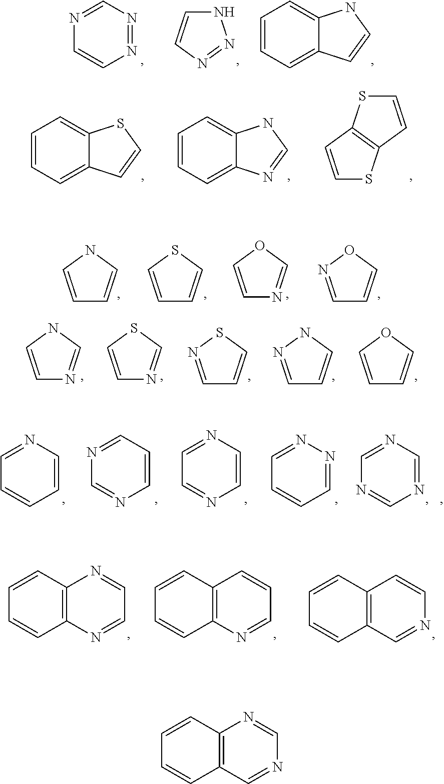

and the like.

A “heteroalicyclic” group or “heterocycloalkyl” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocycloalkyl groups, also referred to as non-aromatic heterocycles, include:

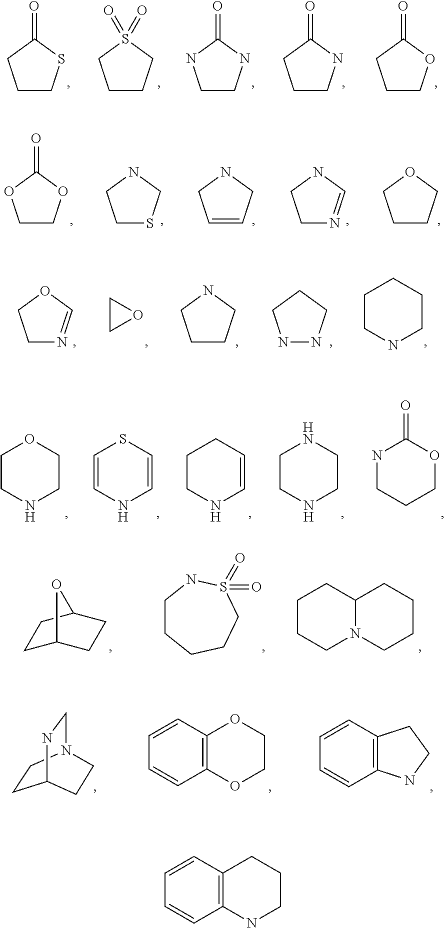

and the like. The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a —CN group.

An “isocyanato” group refers to a —NCO group.

A “thiocyanato” group refers to a —CNS group.

An “isothiocyanato” group refers to a —NCS group.

“Alkoyloxy” refers to a RC(═O)O— group.

“Alkoyl” refers to a RC(═O)— group.

### Methods

in certain embodiments, provided herein is a method treating a proliferative (e.g., hyperproliferative) and/or autoimmune disorder comprising administering to an individual (e.g., a human) in need thereof a therapeutically effective amount of any compound (e.g., a compound of any of Formulas I-V) or composition described herein. In certain embodiments, the method is a method of treating a proliferative disorder and the individual has been diagnosed with, is suspected of having, and/or is predisposed to develop a proliferative disorder. In some embodiments, the proliferative disorder is a hematological proliferative disorder. In certain embodiments, hematological proliferative disorder is, by way of non-limiting example, a lymphoproliferative disorder and a myeloproliferative disorder. In some embodiments, the proliferative disorder is a neoplasia. In certain embodiments, the neoplasia is a hematological neoplasia. In certain embodiments, the proliferative disorder is an autoimmune disease.

In certain embodiments, the neoplasia is, by way of non-limiting example, leukemias, lymphomas, other hematopoietic neoplasias, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosareomas, mast cell tumors, primary hepatic cancers, lung cancers, pancreatic cancers, gastrointestinal cancers, renal cell carcinomas, and metastatic cancers thereof.

In certain embodiments, the neoplasia is a hematological malignancy. Hematological malignancies include, by way of non-limiting example, leukemia/lymphoma, including, but not limited to, B-cell Non-Hodgkin's Lymphoma (NHL), Hodgkin's Disease, and chronic myelogenous leukemia. B-cell Non-Hodgkin's Lymphoma includes, by way of non-limiting example, B cell chronic lymphocytic leukemia/lymphoma (B-CLL), Burkitt's lymphoma (BL), Follicular-like lymphoma (FLL), Diffuse large B-cell lymphoma (DLBCL), multiple myeloma, acute myeloid leukemia (AML), pre-B acute lymphocytic leukemia (ALL), pre-T acute lymphocytic leukemia (ALL), acute promyelocytic leukemia (APL), or refractory leukemia.

Other proliferative disorders include diseases or conditions that are associated with, partially or fully result from, or are characterized by abnormal cell proliferation/growth (dysregulation of cell growth, and typically hyperproliferation) and/or abnormal apoptosis (dysregulation of apoptosis, and typically inhibition of apoptosis). In some embodiments, proliferative disorders partially or fully result from, by way of non-limiting example, a mutation or other dysfunction (e.g., overexpression) of a protooncogene (e.g., Myc) or apoptosis-associated protein (e.g., Bcl-2). For example, polyclonal proliferative diseases, including lymphoproliferative or immunoproliferative disorders are treated in various embodiments described herein. In certain embodiments, such disorders include, by way of non-limiting example, asthma, post-transplant lymphoproliferative disease (PTLD), Castleman's disease, angioimmunoblastic lymphadenopathy, X-linked lymphoproliferative disorders, Epstein Barr Virus (EBV)-associated lymphoproliferative disorder, Wiskott-Aldrich syndrome, ataxia telangiectasia, myeloproliferative disease, thrombocytosis, multiple myeloma, and various autoimmune diseases characterized by lymphoproliferation or lymphadenopathy (e.g., diabetes, Sjögren's syndrome, multiple sclerosis, vitiligo, scleroderma pigmentosa, myasthenia gravis, Multiple Gammopathy of unspecified source (MGUS), Waldentröms' macroglobulinemia, post-transplant lymphoproliferative disease (PTLD), and the like).

In certain embodiments, the methods described herein provide a method of inhibiting the proliferation of, killing and/or inducing apoptosis in cancer stem cells by contacting the cancer stem cell with an effective amount of any compound described herein. As such, in some embodiments, the present invention is useful to treat cancers in which survival of stem cells play a role in the recurrence of the cancer and/or resistance to chemotherapy with at least one other chemotherapeutic agent. In certain embodiments, provided herein is a method of treating a proliferative disorder (e.g., a neoplasia) that is resistant or refractory to at least one anti-cancer or anti-proliferative therapy or agent. In some embodiments, provided herein is a method of treating the recurrence of a proliferative disorder (e.g., a neoplasia). In specific embodiments, provided herein is a method of treating the recurrence of a proliferative disorder (e.g., a neoplasia) following treatment of the proliferative disorder with an anti-proliferative or anti-cancer therapy. In more specific embodiments, the recurrence of the proliferative disorder occurs after the anti-proliferative or anti-cancer therapy caused the proliferative disorder to be in remission (e.g., complete or partial remission) or relapsed.

In some embodiments, a therapeutic compound is administered in a pharmaceutical composition, e.g., one as described herein. In certain embodiments, a therapeutic compound described herein is administered in combination with a second therapeutically effective therapy (e.g., chemotherapy, radiation and/or surgery). In some embodiments, a therapeutic compound described herein is administered before, after, or simultaneously with the second therapeutically effective therapy.

Provided in specific embodiments and non-limiting examples herein is a method of treating a disorder mediated by an oncogene (e.g., a Myc gene), oncoprotein (e.g., a Myc protein) and/or an apoptosis-associated protein (e.g., Bcl-2) by administering to an individual in need thereof a therapeutically effective amount of a compound described herein.

Provided in certain embodiments herein is a method of treating a disorder mediated by the metabolism of glucose by administering any compound described herien, e.g., a compound of Formula II or IV. In some embodiments, a disorder mediated by the metabolism of glucose is a proliferative disorder wherein a cell undergoing abnormal proliferation obtains energy by the metabolism of glucose (e.g., by glycolysis). In certain embodiments, a cell undergoing abnormal proliferation obtains at least 5%, at least 10%, at least 20%, at least 30%, at least 40%, at least 50%, at least 60%, at least 70%, at least 80%, or at least 90% of its energy from the metabolism of glucose.

Provided in some embodiments herein is a method of partially or fully inhibiting the metabolism of glucose by contacting a cell or administering to an individual an effective amount of any compound described herein, e.g., a compound of Formula II or IV. In certain embodiments, provided herein is a method of modulating (e.g., partially or fully inhibiting) the metabolism of glucose in an individual by administering an effective amount of any compound or composition described herein to an individual (e.g., an individual in need of such modulation). In some embodiments, the individual (e.g., human) has been diagnosed with, is suspected of having, or is predisposed to develop a proliferative disorder. In some embodiments, the proliferative disorder is a hematological proliferative disorder. In certain embodiments, hematological proliferative disorder is selected from, by way of non-limiting example, a lymphoproliferative disorder and a myeloproliferative disorder. In some embodiments, the proliferative disorder is cancer. In certain embodiments, the hematological proliferative disorder is a hematological cancer. In certain embodiments, the proliferative disorder is an autoimmune disease.

In some embodiments, provided herein is a method of modulating (e.g., inhibiting) the metabolism of glucose in a cell by contacting the cell with an effective amount of any compound or composition described herein. In some embodiments, the cell is present in an individual (e.g., a human). In certain embodiments, the individual has been diagnosed with, is suspected of having, or is predisposed to develop a proliferative disorder. In some embodiments, the proliferative disorder is a hematological proliferative disorder. In certain embodiments, hematological proliferative disorder is selected from, by way of non-limiting example, a lymphoproliferative disorder and a myeloproliferative disorder. In some embodiments, the proliferative disorder is a neoplasia. In certain embodiments, the hematological proliferative disorder is a hematological cancer. In certain embodiments, the proliferative disorder is an autoimmune disease.

Administration of a compound described herein is achieved in any suitable manner including, by way of non-limiting example, by oral, parenteral (e.g., intravenous, subcutaneous, intramuscular), intranasal, buccal, topical, rectal, or transdermal administration routes.

In some embodiments, an i.v. solution is prepared in a sterile isotonic solution of water for injection and sodium chloride (˜300 mOsm) at a pH of about 11 with a buffer capacity of about 0.006 mol/l/pH unit. In some embodiments, the protocol for preparation of about 100 ml of about 5 mg/ml a first and/or second agent for i.v. infusion is as follows:
a. add about 25 ml of NaOH (about 0.25 N) to about 0.5 g of the active agent and stir until dissolved without heating;
b. add about 25 ml of water for injection and about 0.55 g of NaCl and stir until dissolved;
c. add about 0.1N HCl slowly until the pH of the solution is about 11;
d. the volume is adjusted to about 100 mL;
e. the pH is checked and maintained at about 11; and
f. the solution is subsequently sterilized by filtration through a cellulose acetate (0.22 μm) filter before administration.

In some embodiments, a pharmaceutical composition for oral delivery is prepared by mixing about 100 mg of the active with about 750 mg of a starch. In some embodiments, the mixture is incorporated into an oral dosage unit, such as a hard geletin capsule or coated tablet, which is suitable for oral administration.

In addition, a method for treating any of the diseases or conditions described herein in a subject in need of such treatment, involves administration of a compound described or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to the individual.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein are administered to an individual susceptible to or otherwise at risk of developing a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain instances, wherein following administration of a select dose of one or more compound or compositions described herein and the individual's status does improve, upon the doctor's discretion the administration of a compound or composition described herein is optionally given continuously; alternatively, the dose of drug being administered is optionally temporarily reduced or temporarily suspended for a certain length of time (i.e., a “drug holiday”). In various instances, the length of the drug holiday is selected from between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is optionally from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In certain embodiments, once improvement of an individual's condition has been achieved (following either therapy with a compound or composition described herein; or with an additional therapeutic agent), a maintenance dose of a compound or composition described herein is optionally administered (e.g., to inhibit or prevent the return of cancer stem cells). In certain instances, the dosage or the frequency of administration, or both, is optionally reduced, as a function of the symptoms, to a level at which the improved disease, disorder or condition is retained upon initiation of or during maintenance treatment. In certain instances, however, intermittent treatment is optionally initiated upon any recurrence of symptoms.

In certain embodiments, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In certain embodiments, about 0.02-5000 mg per day, or from about 1-1500 mg per day of a compound described herein is administered. In various embodiments, the desired dose is conveniently be presented in a single dose or in divided doses administered simultaneously (or over a short period of time) or at appropriate intervals, for example as two, three, four or more sub-doses per day.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by pharmaceutical procedures in cell cultures or experimental animals, including, but not limited to, the determinanation of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage faint employed and the route of administration utilized.

Provided in certain embodiments herein is a method of killing, inducing apoptosis in or inhibiting the growth of a cell comprising contacting the cell with an effective amount of a compound or composition described herein, e.g., a compound of any of Formulas I-V. In certain embodiments, the cell is a cell with abnormal proliferation (e.g., compared to a wild type cell of the same lineage). In some embodiments, the cell with abnormal proliferation is an abnormal stem cell or an abnormal progenitor cell. In some embodiments, the cell that is characterized by abnormal proliferation is an abnormal hematopoietic stem cell or a abnormal hematopoietic progenitor cell. In certain embodiments, the cell that is characterized by abnormal proliferation is a cancer stem cell (e.g., a leukemic stem cell). In some embodiments, the cell that is characterized by abnormal proliferation is a cell (e.g., an abnormal hematopoietic stem cell) that partially or fully obtains energy from and/or possesses deregulated glucose metabolism (e.g., glucose catabolism and/or glycogen synthesis). In specific embodiments, the deregulated glucose metabolism of the cell is caused by the overexpression of an oncoprotein (e.g., a Myc oncoprotein). In some embodiments, the cell that is characterized by abnormal proliferation is a cell (e.g., an abnormal hematopoietic stem cell) with increased glucose metabolism (e.g., compared to a wild type cell of the same lineage).

In certain embodiments, the cell that is characterized by abnormal proliferation is present in an individual diagnosed with, is suspected of having, or is predisposed to develop a proliferative disorder. In some embodiments, the proliferative disorder is a hematological proliferative disorder. In certain embodiments, hematological proliferative disorder is selected from, by way of non-limiting example, a lymphoproliferative disorder and a myeloproliferative disorder. In some embodiments, the proliferative disorder is cancer. In certain embodiments, the hematological proliferative disorder is a hematological cancer. In certain embodiments, the proliferative disorder is an autoimmune disease.

In certain embodiments, the cancer is selected from, by way of non-limiting example, leukemias, lymphomas, other hematopoietic neoplasias, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosareomas, mast cell tumors, primary hepatic cancers, lung cancers, pancreatic cancers, gastrointestinal cancers, renal cell carcinomas, and metastatic cancers thereof.

In certain embodiments, the cancer is a hematological malignancy. Hematological malignancies include, by way of non-limiting example, leukemia/lymphoma, including, but not limited to, B-cell Non-Hodgkin's Lymphoma (NHL), Hodgkin's Disease, and chronic myelogenous leukemia. B-cell Non-Hodgkin's Lymphoma includes, by way of non-limiting example, B cell chronic lymphocytic leukemia/lymphoma (B-CLL), Burkitt's lymphoma (BL), Follicular-like lymphoma (FLL), Diffuse large B-cell lymphoma (DLBCL), multiple myeloma, acute myeloid leukemia (AML), pre-B acute lymphocytic leukemia (ALL), pre-T acute lymphocytic leukemia (ALL), acute promyelocytic leukemia (APL), or refractory leukemia.

Other proliferative disorders include diseases or conditions that are associated with, results from, or characterized by abnormal cell proliferation/growth (dysregulation of cell growth, and typically hyperproliferation) and/or abnormal apoptosis (dysregulation of apoptosis, and typically inhibition of apoptosis). In some embodiments, proliferative disorders result from, by way of non-limiting example, a mutation or other dysfunction in an oncogene (e.g., Myc) or apoptosis-associated protein (e.g., Bcl-2). For example, polyclonal proliferative diseases, including lymphoproliferative or immunoproliferative disorders are treated in various embodiments described herein. In certain embodiments, such disorders include, by way of non-limiting example, asthma, post-transplant lymphoproliferative disease (PTLD), Castleman's disease, angioimmunoblastic lymphadenopathy, X-linked lymphoproliferative disorders, Epstein Barr Virus (EBV)-associated lymphoproliferative disorder, Wiskott-Aldrich syndrome, ataxia telangiectasia, myeloproliferative disease, thrombocytosis, multiple myeloma, and various autoimmune diseases characterized by lymphoproliferation or lymphadenopathy (e.g., diabetes, Sjögren's syndrome, multiple sclerosis, vitiligo, scleroderma pigmentosa, myasthenia gravis, Multiple Gammopathy of unspecified source (MGUS), Waldentröms' macroglobulinemia, post-transplant lymphoproliferative disease (PTLD), and the like).

In specific embodiments, provided herein is a method of treating cancer with a compound described herein (e.g., a compound of any of Formulas I-V) and a second cancer therapy (e.g., surgery, radiation and/or an additional chemotherapeutic agent). In some embodiments, the compound described herein is a compound that targets the cancer stem cells of the cancer and the second cancer therapy is a therapy that targets differentiated cancer cells (e.g., a conventional cancer therapy). In some embodiments, a compound described herein is administered before, after, or simultaneously with the second cancer therapy.

### Combinations

In certain instances, it is appropriate to administer at least one therapeutic compound described herein in combination with a second therapeutic agent. By way of example only, if one of the side effects experienced by an individual upon receiving one of the therapeutic compound described herein is nausea, then it is appropriate in certain instances to administer an anti-nausea agent in combination with the initial therapeutic agent. Or, by way of example only, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the individual is enhanced). Or, by way of example only, the benefit experienced by an individual is increased by administering one of the compounds described herein with another therapeutic agent (which also includes a therapeutic regimen) that also has therapeutic benefit. In any case, regardless of the disease, disorder or condition being treated, the overall benefit experienced by the individual is in some embodiments additive of the two therapeutic agents or in other embodiments, the individual experiences a synergistic benefit.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the individual.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified faun, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In some embodiments, the additional therapeutic agent is utilized to achieve remission (partial or complete) of a cancer, whereupon the therapeutic agent described herein (e.g., a compound of any one of Formulas I-V) is subsequently administered. In addition, the combination methods, compositions and formulations are not to be limited to the use of only two agents; the use of multiple therapeutic combinations are also envisioned (including two or more therapeutic compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In addition, the compounds described herein also are optionally used in combination with procedures that provide additional or synergistic benefit to the individual. By way of example only, individuals are expected to find therapeutic and/or prophylactic benefit in the methods described herein, wherein pharmaceutical composition of a compound disclosed herein and/or combinations with other therapeutics are combined with genetic testing to determine whether that individual is a carrier of a gene or gene mutation that is known to be correlated with certain diseases or conditions. In certain embodiments, prophylactic benefit is achieved by administering a therapeutic compound described herein to an individual whose proliferative disorder (e.g., cancer) is in remission (e.g., partial or complete).

In various embodiments, the compounds described herein and combination therapies are administered before, during or after the occurrence of a disease or condition. Timing of administering the composition containing a compound is optionally varied to suit the needs of the individual treated. Thus, in certain embodiments, the compounds are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. The administration of the compounds is optionally initiated within the first 48 hours of the onset of the symptoms, within the first 6 hours of the onset of the symptoms, or within 3 hours of the onset of the symptoms. The initial administration is achieved by any route practical, such as, for example, an intravenous injection, a bolus injection, infusion over 5 minutes to about 5 hours, a pill, a capsule, transdermal patch, buccal delivery, and the like, or combination thereof. In some embodiments, the compound should be administered as soon as is practicable after the onset of a disease or condition is detected or suspected, and for a length of time necessary for the treatment of the disease, such as, for example, from about 1 month to about 3 months. The length of treatment is optionally varied for each subject based on known criteria. In exemplary embodiments, the compound or a formulation containing the compound is administered for at least 2 weeks, between about 1 month to about 5 years, or from about 1 month to about 3 years.

In certain embodiments, therapeutic agents are combined with or utilized in combination with one or more of the following therapeutic agents in any combination: immunosuppressants or anti-cancer therapies (e.g., radiation, surgery or anti-cancer agents).

In specific embodiments, the proliferative disease treated is an autoimmune disease and the additional therapeutic agent is an immunosuppressant. Immunosuppressants include, by way of non-limiting example, tacrolimus, cyclosporin, rapamiein, methotrexate, cyclophosphamide, azathioprine, mercaptopurine, mycophenolate, and FTY720.

In some embodiments, one or more of the anti-cancer agents are proapoptotic agents. Examples of anti-cancer agents include, by way of non-limiting example: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or r1L2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinie acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoelinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stein-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that include alkylating agents, antimetabolites, natural products, or hormones, e.g., nitrogen mustards (e.g., mechloroethamine, cyclophosphamide, chlorambucil, etc.), alkyl sulfonates busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, provided herein is a method of treating lymphoma comprising administering a therapeutically effective amount of a compound described herein in combination with an antibody to CD₂O and/or a CHOP (cyclophosphamide, doxorubicin, vincristine, and prednisone) therapy. In certain embodiments, provided herein is a method of treating leukemia comprising administering a therapeutically effective amount of a compound described herein in combination with ATRA, methotrexate, cyclophosphamide and the like.

### Pharmaceutical Compositions

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a compound described herein, such as, for example, a compound of any of Formulas I-V, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the therapeutic compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage fauns, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein are optionally manufactured in a conventional manner, such as, by way of example only, by means of conventional mixing, dissolving, granulating, dragee-making, levigating, emulsifying, encapsulating, entrapping or compression processes.

In certain embodiments, a pharmaceutical compositions described herein includes one or more compound described herein, e.g., a compound of any of Formulas I-V, as an active ingredient in free-acid or free-base form, or in a pharmaceutically acceptable salt form. In some embodiments, the compounds described herein are utilized as an N-oxide or in a crystalline or amorphous form (i.e., a polymorph). In certain embodiments, an active metabolite or prodrug of a compound described herein is utilized. In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be disclosed herein.

A “carrier” includes, in some embodiments, a pharmaceutically acceptable excipient and is selected on the basis of compatibility with compounds disclosed herein, such as, compounds of any of Formulas I-V, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein are formulated as a dosage form. As such, in some embodiments, provided herein is a dosage form comprising a compound described herein, e.g., a compound of any of Formulas I-V, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using coating procedures, such as those described in Remington's Pharmaceutical Sciences, 20th Edition (2000), a film coating is provided around the formulation of the compound of any of Formula I-V. In one embodiment, a compound described herein is in the form of a particle and some or all of the particles of the compound are coated. In certain embodiments, some or all of the particles of a compound described herein are microencapsulated. In some embodiment, the particles of the compound described herein are not microencapsulated and are uncoated.

In certain embodiments, the pharmaceutical composition described herein is in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. Aqueous suspension compositions are optionally packaged in single-dose non-reclosable containers. In some embodiments, multiple-dose re-closeable containers are used. In certain instances, multiple dose containers comprise a preservative in the composition. By way of example only, formulations for parenteral injection are presented in unit dosage form, which include, but are not limited to ampoules, or in multi-dose containers, with an added preservative.

### Screening Process

Provided in certain embodiments herein are processes and kits for identifying compounds suitable for treating proliferative disorders and/or killing, inducing apoptosis in or inhibiting the proliferation of a cell. In certain embodiments, provided herein are processes and kits for identifying compounds that selectively inhibit the growth of, kill, induce apoptosis in, or a combination thereof in abnormally proliferating stem cells by:
a. providing a plurality of conditionally immortalized stem cells and a plurality of abnormally proliferating stem cells;
b. contacting the plurality of conditionally immortalized stem cells with a compound;
c. contacting the plurality of abnormally proliferating stem cells with the compound;
d. detecting or measuring the affect of the compound on viability of the plurality of conditionally immortalized stem cells and the plurality of abnormally proliferating stem cells; and
e. comparing the effect of the compound on viability of the plurality of conditionally immortalized stem cells to the effect of the compound on the viability of the plurality of abnormally proliferating stem cells.

In certain embodiments, the plurality of conditionally immortalized stem cells comprises a plurality of conditionally immortalized hematopoietic stem cells.

### Conditionally Immortalized Cells

In some embodiments, the assay comprises providing a plurality of conditionally immortalized stem cells. In some embodiments, conditional immortality is conferred by modifying (e.g., up-regulating) the expression of a proto-oncogene (e.g., Myc, Notch-1, Akt, hTERT), and/or an anti-apoptotic gene (i.e., a gene that encodes a polypeptide that inhibits apoptosis; e.g., bcl-2, bcl-x, mcl-1). In some embodiments, the expression of a proto-oncogene (e.g., Myc, Notch-1, Akt, hTERT), and/or an anti-apoptotic gene (i.e., a gene that encodes a polypeptide that inhibits apoptosis; e.g., bcl-2, bcl-x, mcl-1) is modulated by any suitable manner (e.g., by use of tetracycline controlled transcriptional activation, or by use of a fusion protein). In certain embodiments, the conditionally immortalized stem cells are those set forth or prepared by a method set forth in U.S. 2007/0116691, which is hereby incorporated by reference for such disclosures.

In some embodiments, a conditionally immortalized stem cells comprises a pro-apoptotic polypeptide (i.e., a polypeptide the activity of which promotes, induces, and/or initiates apoptosis). In some embodiments, the pro-apoptotic polypeptide is a member of the Bcl-2 protein family (e.g. Bim, PUMA, NOXA, Bak, and Bax). In some embodiments, the pro-apoptotic polypeptide has been modified by any suitable manner (e.g., by use of CRE or FLP, or tetracycline controlled transcriptional activation). In some embodiments, the pro-apoptotic polypeptide has been modified such that the expression of the pro-apoptotic polypeptide is partially or fully down-regulated. In some embodiments, the pro-apoptotic polypeptide has been modified such that the pro-apoptotic polypeptide is not expressed.

In some embodiments, a conditionally immortalized cell line described herein is homogenous in phenotype and exhibits the phenotype of long-term hematopoietic stem cells (lt-HSC) that provide all long term reconstitution in mice, and are easily recovered after freezing, retaining their original phenotype. In some embodiments, a conditionally immortalized cell line described herein gives rise to additional long-term HSCs as well as all of the lineages normally derived from HSCs. In some embodiments, the resulting cell lines have a surface phenotype that resembles normal lt-HSC cell lines, but give rise to leukemias that resemble AMLs. In some embodiments, a conditionally immortalized cell line described herein is able to give rise to a leukemia that has a surface phenotype different that the LSC, and retain some of its pluripotency, as evidenced by their ability to give rise to some of the hematopoietic lineages that emerge from normal HSCs.

In some embodiments, the proto-oncogene and/or the gene that encodes a polypeptide that inhibits apoptosis of the plurality of cells is excised from the genome of the stem cell. In some embodiments, excision of the proto-oncogene and/or the gene that encodes a polypeptide that inhibits apoptosis of the plurality of cells is achieved in any suitable manner including, via bacterial recombinases (e.g., Cre or Flp).

### Tetracycline Controlled Transcriptional Activation

In some embodiments, the transcription of a proto-oncogene and/or anti-apoptotic gene is controlled by tetracycline or an analog thereof (e.g., doxycycline).

In some embodiments, the genome of the conditionally immortalized stem cell is modified such that the proto-oncogene and/or anti-apoptotic gene is under the control of (i.e., downstream from) a tetO operator. In some embodiments, the genome of the conditionally immortalized stem cell is further modified such the genome comprises the sequence for a tetracycline transactivator (tTA) (i.e., a fusion of a TetR sequence and a VP16 sequence). In some embodiments, the genome of a stem cell is transformed/transfected by any suitable manner (e.g. by nucleofection, electroporation, heat shock, magnetofection; or by the use of calcium phosphate, dendrimers, cationic polymers, liposomes, or a gene gun). In the absence of tetracycline, a tTA protein binds at a tetO operator and activates a promoter. In certain instances, the activation of the promoter induces expression of the proto-oncogene and/or anti-apoptotic gene and confers immortality. When the cells are contacted with tetracycline, or a derivative thereof, the tetracycline (or derivative thereof) inhibits the binding of the tTA protein to the tetO operator and the cells are not immortalized.

In some embodiments, the genome of the conditionally immortalized stem cell is modified such the genome comprises the sequence for a reverse tetracycline transactivator (rtTA). In some embodiments, the genome of a stem cell is transformed/transfected by any suitable manner (e.g. by nucleofection, electroporation, heat shock, magnetofection; or by the use of calcium phosphate, dendrimers, cationic polymers, liposomes, or a gene gun). In the presence of doxycycline, the rtTA protein binds at a tetO operator and activates a promoter coupled to the tetO operator. In certain instances, the activation of the promoter induces expression of the proto-oncogene and/or anti-apoptotic gene and confers immortality. In the absence of doxycycline the rtTA protein cannot bind to the tetO operator and the cells are not immortalized.

In some embodiments, the genome of a stem cell is transformed/transfected (e.g. by use of a retrovirus, by nucleofection, electroporation, heat shock, magnetofection; or by the use of calcium phosphate, dendrimers, cationic polymers, liposomes, or a gene gun) with a nucleotide sequence encoding a tetracycline controlled transcription activation complex described herein.

### Fusion Proteins

In some embodiments, the proto-oncogene and/or anti-apoptotic gene is fused to a receptor (i.e., it is a fusion protein). In some embodiments, the receptor is an estrogen receptor (ER). In some embodiments, the fusion protein is MYC-ER. In certain instances, contacting a cell with a ligand (e.g., tamoxifen or 4-hydroxytamoxifen if the receptor is an ER) for the receptor induces expression of the proto-oncogene and/or anti-apoptotic gene and the cell line is immortalized. In the absence of a ligand for the receptor, expression of the proto-oncogene and/or anti-apoptotic gene is not induced and the cells are not immortalized.

In some embodiments, the genome of a stem cell is transformed/transfected (e.g. by use of a retrovirus, by nucleofection, electroporation, heat shock, magnetofection; or by the use of calcium phosphate, dendrimers, cationic polymers, liposomes, or a gene gun) with a nucleotide sequence encoding a fusion protein described herein. In certain embodiments, a fusion protein described herein further comprises a transduction domain, e.g., Tat or Vpr. In some embodiments, a stem cell is contacted with a fusion protein described herein.

### Assay

In some embodiments, the assay comprises immortalizing a plurality of cells (e.g., contacting a plurality of cells from a MYC-ER cell line with an ER ligand, removing tetracycline from a plurality of cells from a tTA cell line, or contacting a plurality of cells from an rtTA cell line with doxycycline). In some embodiments, after a desired density of cells has been achieved, the assay comprises unimmortalizing the plurality of cells (e.g., removing and/or withdrawing the ER ligand in a MYC-ER cell line, adding tetracycline to a tTA line, or removing doxycycline from an rtTA cell line). In certain embodiments, the assay comprises contacting a plurality of conditionally immortalized cells with a compound under conditions that do not confer immortality.

In some embodiments, before a plurality of conditionally immortalized cells is contacted with the compound, the proto-oncogene and/or the gene that encodes a polypeptide that inhibits apoptosis of the cell is excised from the plurality of conditionally immortalized stem cell.

In some embodiments, for viability assays, the cell lines are passed 24-36 hours prior to use in the assay, in order to test for sensitivity to specific drugs with cells in log-phase growth. In some embodiments, cells are plated in 96-well flat bottom plates (Greiner, Switzerland), at a concentration of 10⁴ cells for the leukemic stem cell lines and normal hematopoietic stem cell lines, or 10⁵ for the primary human fetal cord blood cells. In some embodiments, cells are plated in a final volume of 200 μl containing RPMI-1640 growth medium, supplemented as described above. In some embodiments, cells are either plated in medium alone, or medium containing a drug of interest. All drugs are tested in 11 different concentrations in order to derive sensitivity curves.

In some embodiments, the cells are cultured with the specific drug concentration for 24 hours at 37° C. in a 5% CO₂ atmosphere. In some embodiments, after culturing the cells under the experimental conditions for 23 hours, the wells are supplemented with 10 μl of the CellTiter 96 Aqueous Non-radioactive Cell Proliferation MTS reagent (Promega, Madison Wis.), and incubated in a 37° C. incubator (5% CO₂) for 60 minutes. All plates are analyzed using a plate reader (Molecular Devices) using a filter set for O.D. 405. All values are used to derive standard errors, and compared among the different independent assays.

In some embodiments, the abnormally proliferating stem cell is an abnormal hematopoietic stem cell. In certain embodiments, the abnormally proliferating stem cell is a cancer stem cell (e.g., a leukemic stem cell). In certain embodiments, the abnormally proliferating stem cell is a hematapoietic cancer stem cell. In certain embodiments, the abnormal proliferation of the abnormally proliferating stem cell described herein is caused, at least in part, by a loss of regulation of or an unregulated protooncogene or oncogene; by the overexpression of an oncoprotein (as used herein, oncoprotein includes protooncoprotein); by the overexpression of an apoptosis inhibiting polypeptide, or a combination thereof.

Furthermore, in some embodiments, the abnormally proliferating stem cell utilized in a method of identifying a compound that selectively inhibit the growth of, kill, induce apoptosis in, or a combination thereof in an abnormally proliferating stem cell (e.g., cancer stem cell) is prepared by altering the genotype of the conditionally immortalized stem cell utilized. For example, Example 1 sets forth a method of preparing leukemic stem cell lines.

In certain embodiments, detecting or measuring of the effect of the compound on the viability of the conditionally immortalized stem cell and the abnormally proliferating stem cell is achieved in any suitable manner including, by way of non-limiting example, any assays for autophagy and/or necrosis, 7AAD staining, a GFP viability assay, Annexin V surface staining, TUNEL assay, MTT or MTS assay, mitochondrial potential assay, Caspase 9, 10, 3, 6 or 8 cleavage assays (fluorometric or otherwise), H³-thymidine incororation assay, CFSE, or equivalent dyes for proliferation, or a combination thereof. As used herein, viability includes, e.g., the ability to survive and the ability proliferate.

In some embodiments, analysis of apoptosis is by flow cytometry. In some embodiments, cells are cultured (in triplicate) with the specific drug concentration for 24 hours at 37° C. in a 5% CO₂ atmosphere. In some embodiments, cell suspensions obtained from cultured cells are washed twice in FACS buffer, incubated for 20 minutes with 4 μM 7-aminoactinomycin-D (7AAD, Calbiochem), and washed with FACS buffer. In some embodiments, stained cells are resuspended in PBS and analyzed immediately on a flow cytometer.

In some embodiments, the process described herein is also utilized to identifying compounds that inhibit the growth of, kill, induce apoptosis in, or a combination thereof in abnormally proliferating cells (e.g., a cancer, leukemia or tumor cell), but do not substantially affect the viability of a normal stem cell. In such embodiments, the process is as described herein, but instead of utilizing abnormally proliferating stem cells, abnormally proliferating cells, e.g., of an established tumor line, are utilized.

In some embodiments, provided herein is a compound identified by any process described herein for identifying a compound suitable for treating proliferative disorders and/or killing, inducing apoptosis in or inhibiting the proliferation of a cell. Furthermore, provided herein are methods of treating the disorders described herein with a compound so identified.

In certain embodiments, kits for identifying compounds suitable for treating proliferative disorders and/or killing, inducing apoptosis in or inhibiting the proliferation of a cell comprise (i) a plurality of conditionally immortalized stem cells, and (ii) a plurality of abnormally proliferating stem cells, wherein such cells are as described in the screening process set forth herein.

### EXAMPLES

The following example describes compounds that preferentially inhibit the growth of, kill, and/or induce apoptosis in abnormally proliferating cells (e.g., cancer stem cells). In certain instances, these compounds are suitable for the treatment of hematological diseases including, but not limited to, cancer, autoimmune disorders and hyperproliferative disorders. These examples are for illustrative purposes only and are non-limiting embodiments. Many modifications, equivalents, and variations of the present invention are possible in light of the above teachings, therefore, it is to be understood that within the scope of the appended claims, the invention may be practiced other than as specifically described.

### Example 1

### Preparation of Hematopoetic Stem Cell Line and Leukemic Stem Cell Lines

Normal conditionally immortalized stem cell lines (ctlt-HSC cell lines) are prepared from 5FU treated mice were transduced with retroviruses encoding MYC-ER and Bcl-2 and transferred into lethally irradiated recipient mice (1200 rads). Ten days later, weekly intraperitoneal injections of 1 mg/mouse of 4-hydroxytamoxifen (4-OHT) emulsified in oil are initiated to activate MYC function. Within four weeks, recipients of young transduced stem cells developed tumors. The tumors are harvested from bone marrow, spleen and lymph nodes and cultured in vitro with 4-OHT and a stem cell growth factor cocktail (IL-6, IL-3 and stem cell factor (SCF)). These cell lines are homogenous in phenotype and exhibit the phenotype of long-term hematopoietic stem cells (lt-HSC) that provide all long term reconstitution in mice, and are easily recovered after freezing, retaining their original phenotype. Importantly, these cell lines give rise to additional long-term HSCs as well as all of the lineages normally derived from HSCs. The leukemic stem cell line, ABM42-C31 is a clone established from normal ctlt-HSC cell line produced by limiting dilution without any helper or feeder cells. The resulting cell lines have a surface phenotype that resembles normal lt-HSC cell lines, but give rise to leukemias that resemble AMLs. In addition, some of the other leukemia stem cell lines we have established in this way are able to give rise to a leukemia that has a surface phenotype different that the LSC, and retain some of their pluripotency, as evidenced by their ability to give rise to some of the hematopoietic lineages that emerge from normal HSCs.

### Example 2

### Viability Based Drug Screen

Leukemic stem cell lines and normal hematopoetic stem cell lines are separately maintained in cultures as described above. For viability assays, the cell lines are passed 24-36 hours prior to use in the assay, in order to test for sensitivity to specific drugs with cells in log-phase growth. Cells are plated in 96-well flat bottom plates (Greiner, Switzerland), at a concentration of 10⁴ cells for the leukemic stem cell lines and normal hematopoietic stem cell lines, or 10⁵ for the primary human fetal cord blood cells. Cells are plated in a final volume of 200 μl containing RPMI-1640 growth medium, supplemented as described above. Cells are either plated in medium alone, or medium containing a drug of interest. All drugs are tested in 11 different concentrations in order to derive sensitivity curves. The individual conditions were set up in triplicate wells, and at least three independent assays are performed to validate a specific observation.

The cells are cultured with the specific drug concentration for 24 hours at 37° C. in a 5% CO₂ atmosphere. After culturing the cells under the experimental conditions for 23 hours, the wells are supplemented with 10 μl of the CellTiter 96 Aqueous Non-radioactive Cell Proliferation MTS reagent (Promega, Madison Wis.), and incubated in a 37° C. incubator (5% CO₂) for 60 minutes. All plates are analyzed using a plate reader (Molecular Devices) using a filter set for O.D. 405. All values are used to derive standard errors, and compared among the different independent assays.

### Flow Cytometric Analysis of Apoptosis Induction

For analysis of apoptosis in leukemic stem cells or in normal hematopoietic stem cell lines, cells are cultured (in triplicate) with the specific drug concentration for 24 hours at 37° C. in a 5% CO₂ atmosphere. Cell suspensions obtained from cultured cells are washed twice in FACS buffer, incubated for 20 minutes with 4 μM 7-aminoactinomycin-D (7AAD, Calbiochem), and washed with FACS buffer. Stained cells are resuspended in PBS and analyzed immediately on a flow cytometer.

### Therapeutic Trials

Groups of 3-5 mice are utilized for each of the experimental protocols. Transplantation of tumors is done by injecting 10³ cells intravenously into cohorts of sub-lethally irradiated (450R)C57/BL6/Rag −/− mice ranging in age from 4-6 weeks. The mice are monitored daily for clinical signs of disease. These clinical signs included externally evident splenomegaly, a hunched posture and reduce mobility, labored breathing, dehydration, scruffy fur and an ascending paralysis in a minority of cases. The transplant recipient mice are monitored until mice exhibited clinical signs of disease (approximately 15 days following transplant of a leukemic stem cell clone). The mice then receive daily injections of the indicated agents for 7 days. Mice are held indefinitely to ascertain rates of survival.

### Example 3

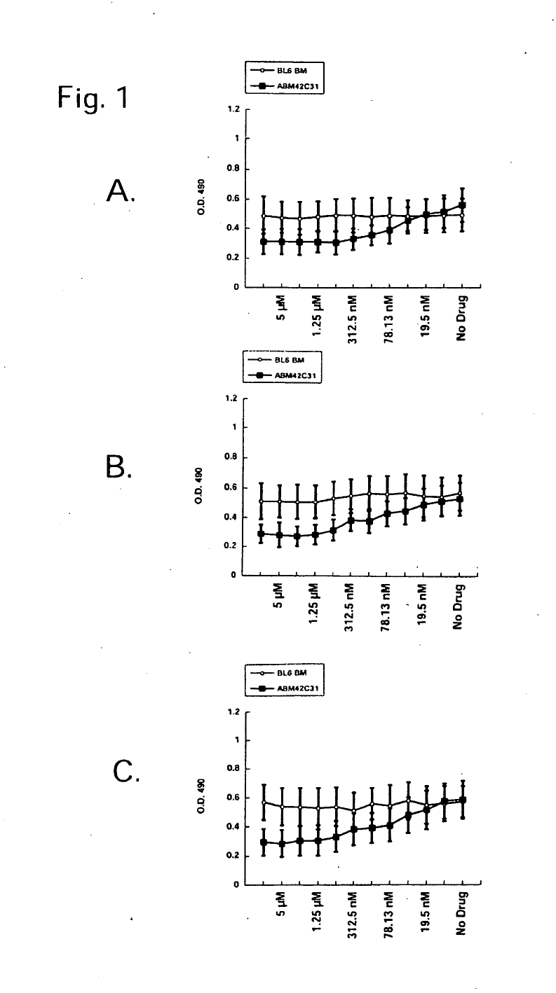

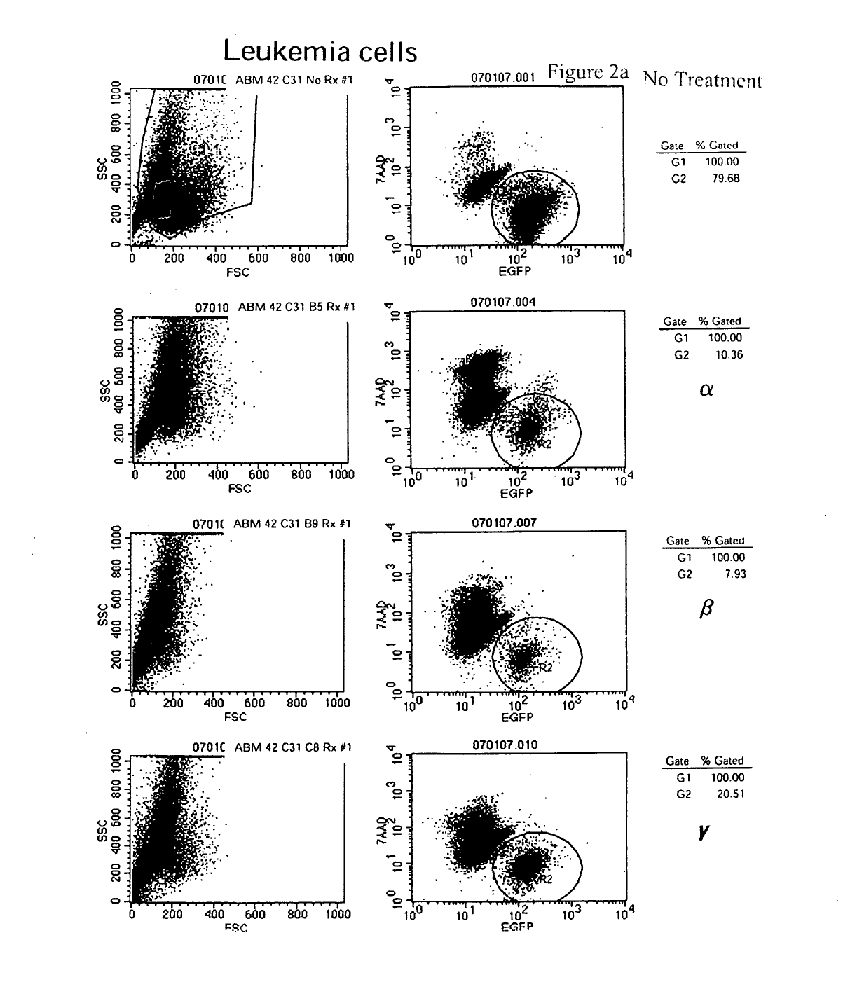

4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione preferentially inhibit murine leukemic stem cell viability vs. normal murine hematopoietic stem cell line. These compounds are screened for the ability to inhibit leukemic stem cell viability but not affect normal hematopoietic stem cells by incubating the compounds with cells using serial two-fold dilutions starting from 10 μM stocks. These compounds preferentially inhibited viability of the leukemic stem cell clone ABM42 C31 but not the normal murine hematopoietic stem cell line “BL/6 BM” (FIG. 1).

### Example 4

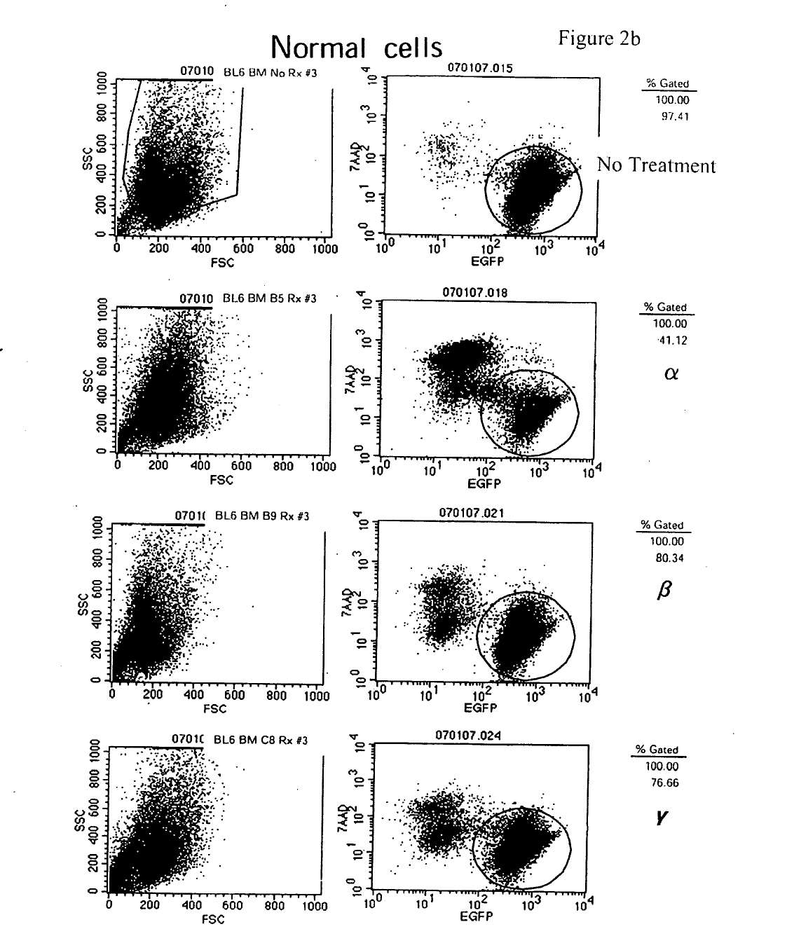

4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione preferentially kill murine leukemic stem cell viability vs. normal murine hematopoietic stem cell line. These compounds are screened to determine whether the effect of the compounds on reduced viability was due to inhibiting proliferation, or direct killing through apoptosis. The cells are analyzed by flow cytometric analysis for the exclusion of 7-aminoactinomycin-D (7AAD). At a concentration of 10 μM, cells are incubated in a 5% CO₂ atmosphere for 24 h. After two washes in 1×PBS the cells are incubated with 4 μM 7-aminoactinomycin-D (7AAD) for 20 min. FIG. 2 illustrates that 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione induce apoptosis preferentially in leukemic stem cells vs. noinial marine hematopoetic stem cell lines as evidenced by an increase in 7AAD positive(y-axis)/GFP negative(x-axis) and a decrease in 7AAD negative/GFP positive expression α-axis).

### Example 5

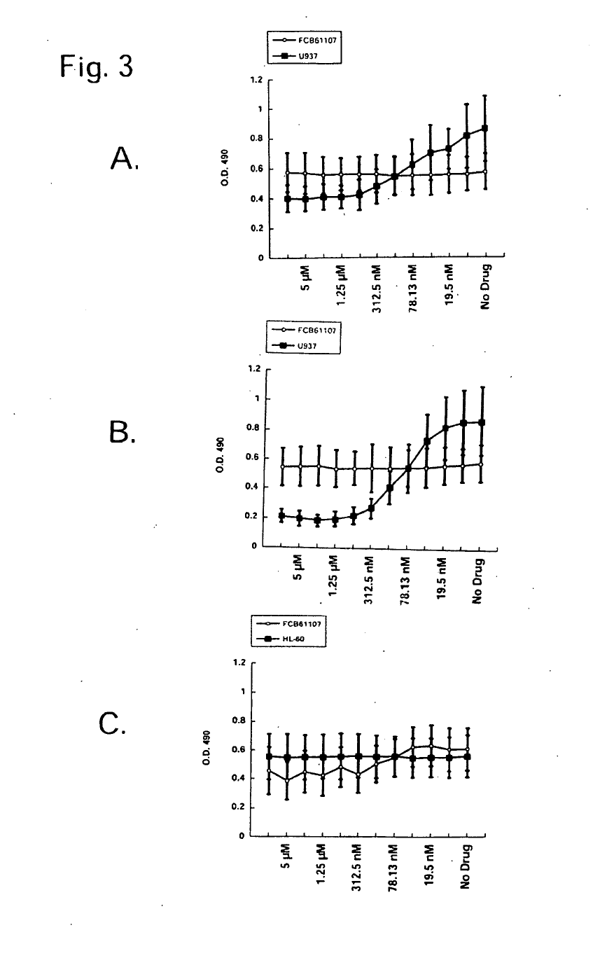

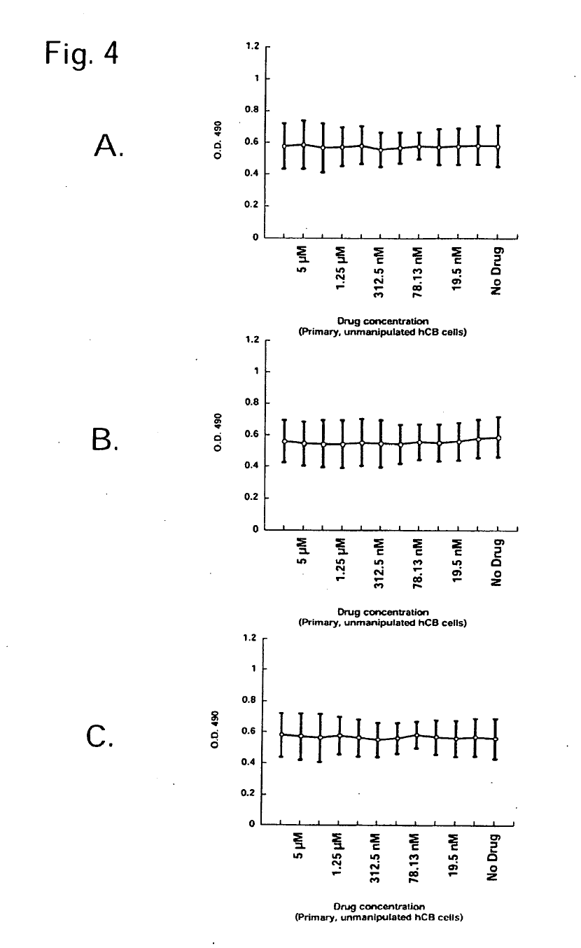

4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione preferentially inhibit human leukemic cell line viability vs. normal human stem cell line and unmanipulated human fetal cord blood. These compounds are incubated with cells using serial two-fold dilutions beginning with 10 μM. These compounds preferentially inhibited viability of the leukemic cell lines but not the normal human hematopoietic stem cell line “FCB61107” (FIG. 3). Importantly, at the drug concentrations tested, there is no decrease in viability of primary unmanipulated fetal cord blood cultures (FIG. 4).

### Example 6

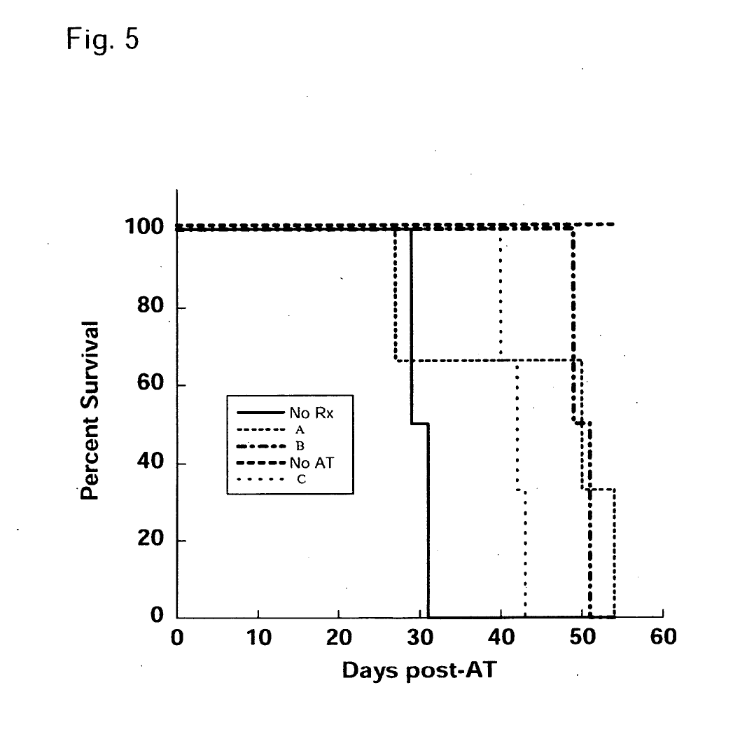

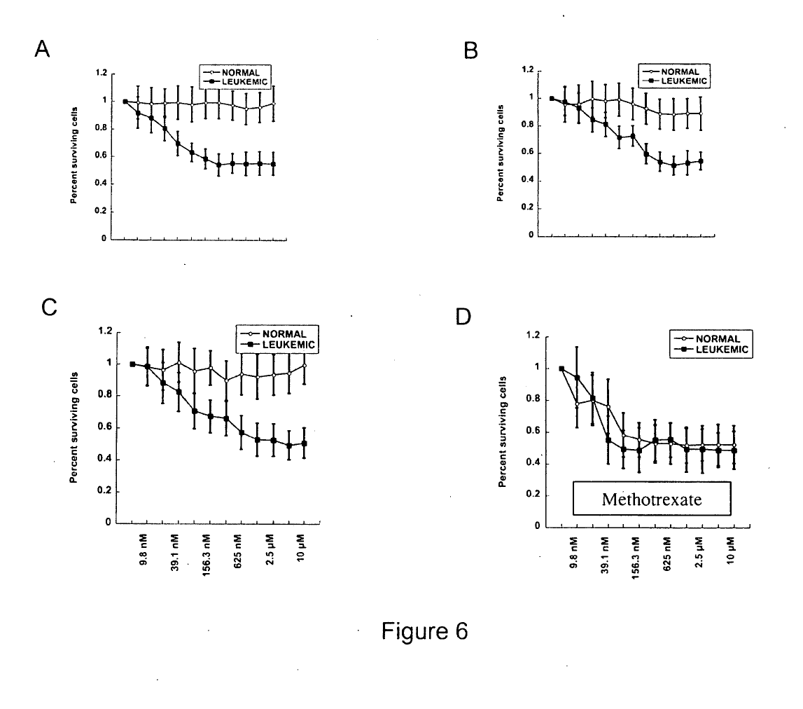

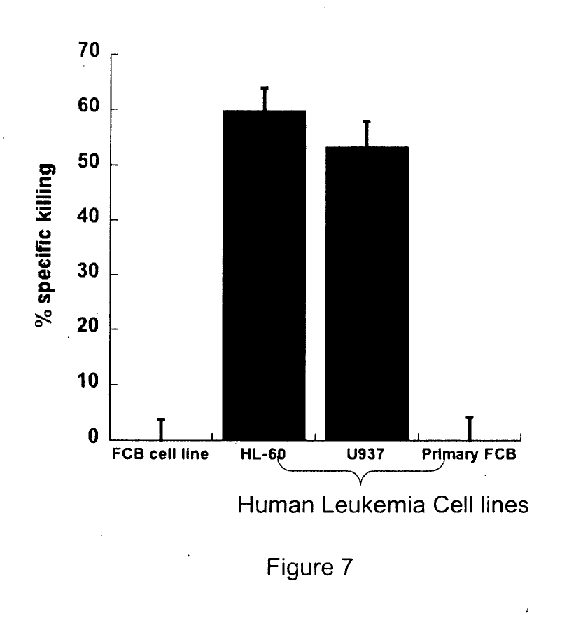

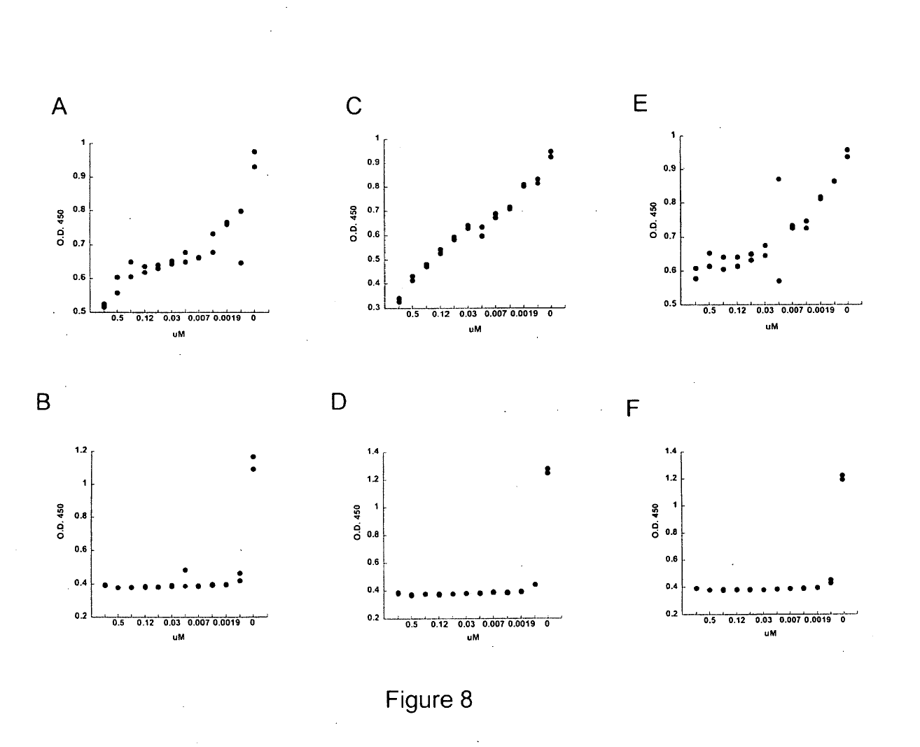

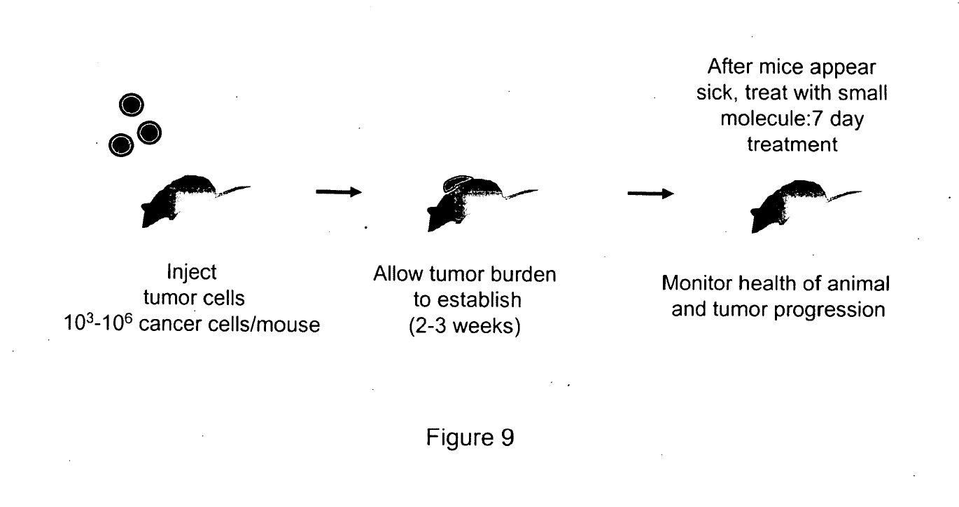

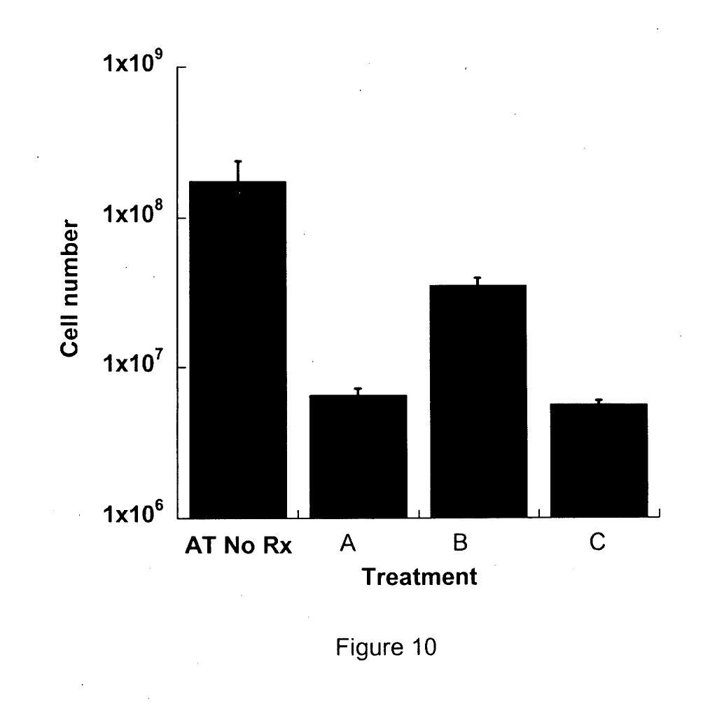

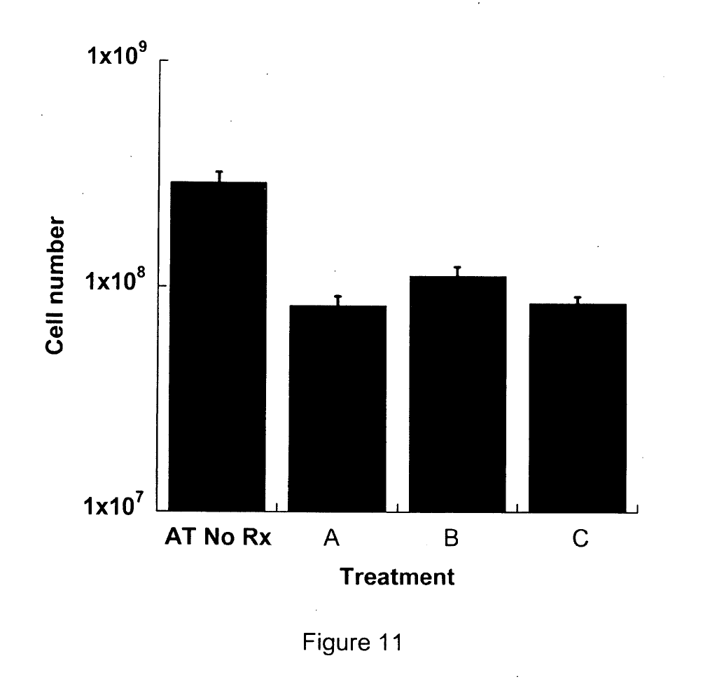

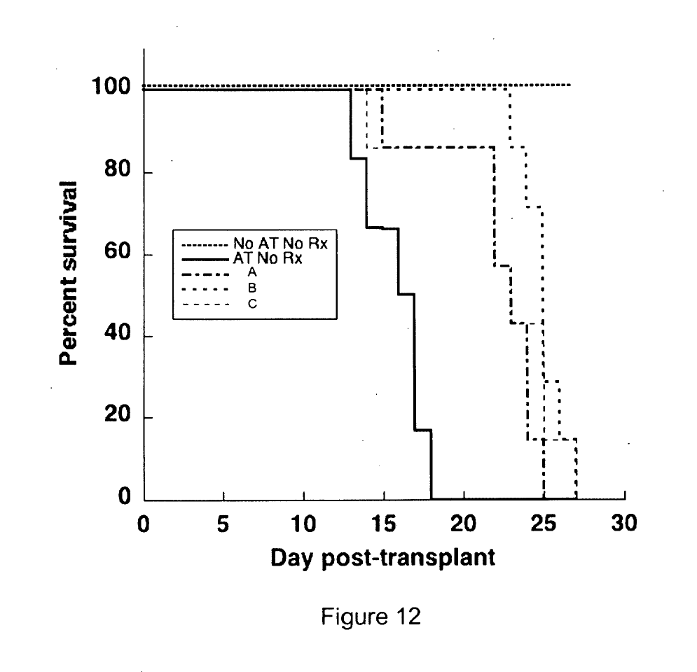

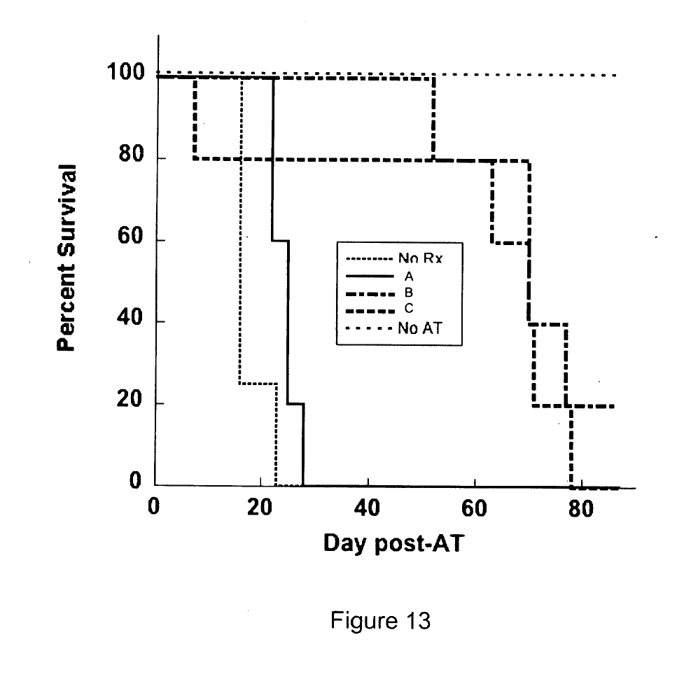

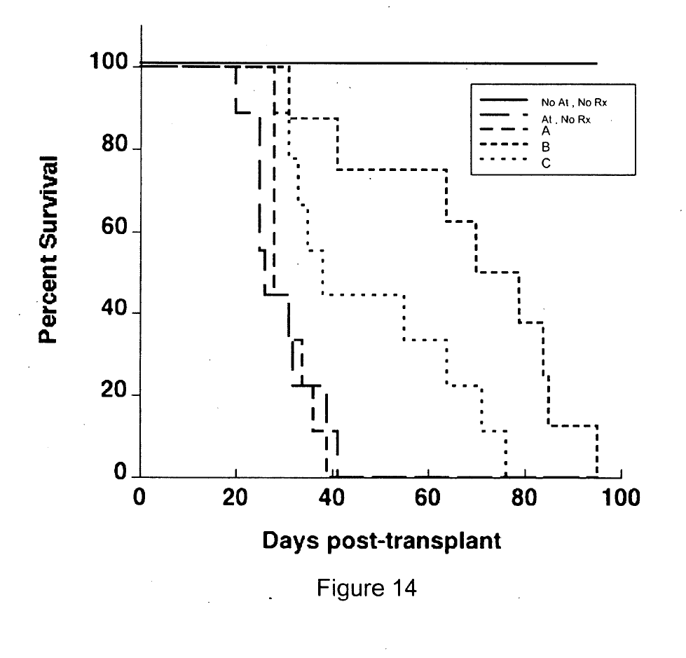

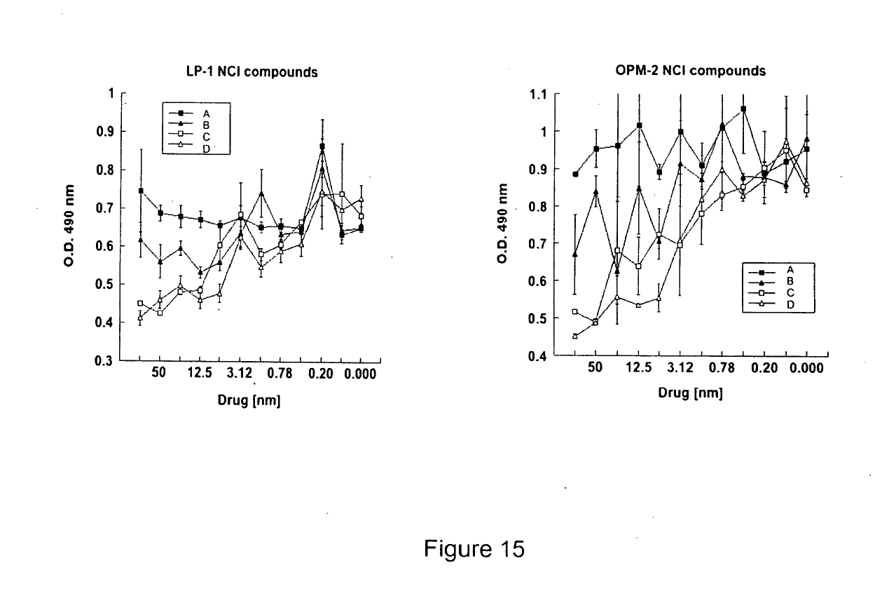

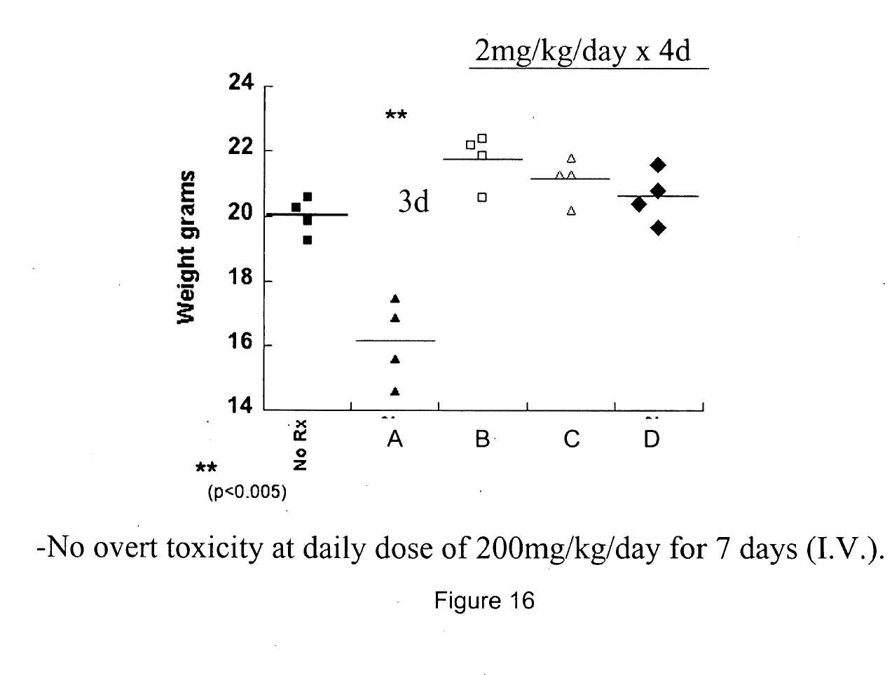

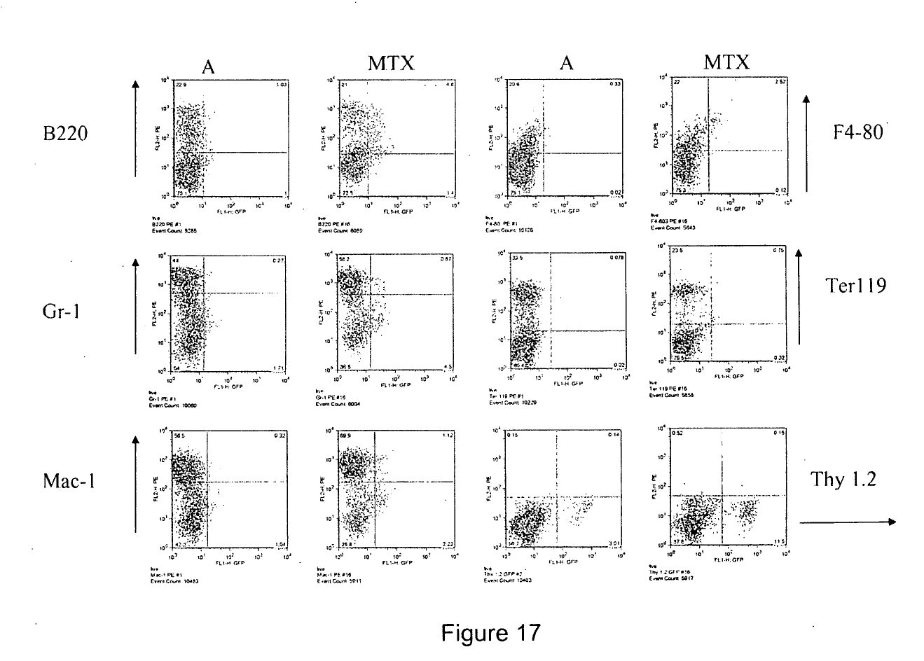

4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione increase survival of mice transplanted with leukemic stem cells. Into cohorts of sublethally irradiated mice are transplanted 10³ ABM 42 C31 leukemic stem cells. Treatment of the mice when they develop clinical signs of disease (hunched posture, rapid breathing, scruffled fur at approximately 15d post-transplant). Mice receive 7 daily injections of 250 ul XPBS solutions containing 10 uM concentrations of 4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione. After the 7^{th} day the mice are monitored for survival. FIG. 5 illustrates that administration of the compounds delay mortality in a pre-clinical model of AML disease.

### Example 7

4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione increase survival of mice transplanted with leukemic stem cells. Immune deficient mice that are reconstituted with the murine hematopoetic stem cell lines are injected with 10,000× the effective in vitro concentration of a drug. Briefly, mice are given 7 daily injections of 1 mM concentrations of one of 4-Amino-7-[(2R,3S,4S,5R)-3,4-dihydroxy-5-(hydroxymethyl)oxolan-2-yl]-6-hydrazinylpyrrolo[5,4-d]pyrimidine-5-carboxamide, 2-[(2-chloro-4-nitrophenanthridin-6-yl)amino]ethanol, and 5-(anthracen-1-ylmethylidene)-2-sulfanylidene-1,3diazinane-4,6-dione. After the 7^{th} day the mice are monitored for overt side-effects and survival. At this dose and regimen, none of the mice died.

### Example 8

Human Clinical Trial of the Safety and/or Efficacy of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide (or a pharmaceutically acceptable salt thereof) therapy

Objective: To determine the safety and pharmacokinetics of administered 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide.

Study Design: This will be a Phase I, single-center, open-label, randomized dose escalation study followed by a Phase II study in leukemian individuals. Individuals should not have had exposure to 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide prior to the study entry. Individuals must not have received treatment for their cancer within 2 weeks of beginning the trial. Treatments include the use of chemotherapy, hematopoietic growth factors, and biologic therapy such as monoclonal antibodies. The exception is the use of hydroxyurea for individuals with WBC>30×103/μL. This duration of time appears adequate for wash out due to the relatively short-acting nature of most anti-leukemia agents. Individuals must have recovered from all toxicities (to grade 0 or 1) associated with previous treatment. All subjects are evaluated for safety and all blood collections for pharmacokinetic analysis are collected as scheduled. All studies are performed with institutional ethics committee approval and individual consent.

Phase I: Individuals receive intravenous 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide daily for 5 consecutive days or 7 days a week. Doses of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide may be held or modified for toxicity based on assessments as outlined below. Treatment repeats every 28 days in the absence of unacceptable toxicity. Cohorts of 3-6 individuals receive escalating doses of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide until the maximum tolerated dose (MTD) for the 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide is determined. The MTD is defined as the dose preceding that at which 2 of 3 or 2 of 6 individuals experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Individuals receive 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide as in phase I at the MTD determined in phase I. Treatment repeats every 6 weeks for 2-6 courses in the absence of disease progression or unacceptable toxicity. After completion of 2 courses of study therapy, individuals who achieve a complete or partial response may receive an additional 4 courses. Individuals who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide. Venous blood samples (5 mL) for determination of serum concentrations are obtained at about 10 minutes prior to dosing and at approximately the following times after dosing: days 1, 2, 3, 4, 5, 6, 7, and 14. Each scrum sample is divided into two aliquots. All scrum samples are stored at −20° C. Scrum samples are shipped on dry ice.

Pharmacokinetics: Individuals undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Individual Response to Therapy: individual response is assessed with bone marrow aspiration/biopsy and is performed prior to beginning the study and at the end of the first cycle, with additional bone marrow aspiration/biopsy performed every four weeks or at the end of subsequent cycles. Individuals also undergo biopsy to assess changes in progenitor cancer cell phenotype and clonogenic growth by flow cytometry, and for changes in cytogenetics by FISH or detection of chromosomal translocations by TaqMan PCR as a means to measure tumor burden. After completion of study treatment, individuals are followed periodically for 4 weeks.

An alternative approach includes testing AML individuals that have no real therapeutic options that have demonstrated efficacy. On humane ground, the request for an expedited Phase I/II combined trial is optionally proposed. In this instance, the initial dose escalation studies necessary to determine MTD in a Phase I is also accompanied by monitoring for clinical outcomes, rather than having to wait for Phase II.

### Example 9

Human Clinical Trial of the Safety and/or Efficacy of 2-[(2-chloro-4-nitrophenanthridin-6-yl)amino]ethanol (or a pharmaceutically acceptable salt thereof) therapy

Objective: To determine the safety and pharmacokinetics of administered 2-[(2-chloro-4-nitrophenanthridin-6-yl)amino]ethanol.

Study Design: This will be a Phase I, single-center, open-label, randomized dose escalation study followed by a Phase II study in leukemian individuals. Individuals should not have had exposure to 2-[(2-chloro-4-nitrophenanthridin-6-yl)amino]ethanol prior to the study entry. Individuals must not have received treatment for their cancer within 2 weeks of beginning the trial. Treatments include the use of chemotherapy, hematopoietic growth factors, and biologic therapy such as monoclonal antibodies. The exception is the use of hydroxyurea for individuals with WBC>30×103/μL. This duration of time appears adequate for wash out due to the relatively short-acting nature of most anti-leukemia agents. Individuals must have recovered from all toxicities (to grade 0 or 1) associated with previous treatment. All subjects are evaluated for safety and all blood collections for pharmacokinetic analysis are collected as scheduled. All studies are performed with institutional ethics committee approval and individual consent.

Phase I: Individuals receive intravenous 2-[(2-chloro-4-nitrophenanthridin-6-yl)amino]ethanol daily for 5 consecutive days or 7 days a week. Doses of 2-[(2-chloro-4-nitrophenanthridin-6-yl)amino]ethanol may be held or modified for toxicity based on assessments as outlined below. Treatment repeats every 28 days in the absence of unacceptable toxicity. Cohorts of 3-6 individuals receive escalating doses of 2-[(2-chloro-4-nitrophenanthridin-6-yl)amino]ethanol until the maximum tolerated dose (MTD) for the 2-[(2-chloro-4-nitrophenanthridin-6-yl)amino]ethanol is determined. The MTD is defined as the dose preceding that at which 2 of 3 or 2 of 6 individuals experience dose-limiting toxicity. Dose limiting toxicities are deteii lined according to the definitions and standards set by the National Cancer Institute (NCI) Common Terminology for Adverse Events (CTCAE) Version 3.0 (Aug. 9, 2006).

Phase II: Individuals receive 2-[(2-chloro-4-nitrophenanthridin-6-yl)amino]ethanol as in phase I at the MTD determined in phase I. Treatment repeats every 6 weeks for 2-6 courses in the absence of disease progression or unacceptable toxicity. After completion of 2 courses of study therapy, individuals who achieve a complete or partial response may receive an additional 4 courses. Individuals who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 2-[(2-chloro-4-nitrophenanthridin-6-yl)amino]ethanol. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Individuals undergo plasma/serum sample collection for pharmacokinetic evaluation before beginning treatment and at days 1, 2, 3, 4, 5, 6, 7, and 14. Pharmcokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Individual Response to therapy: Individual response is assessed with bone marrow aspiration/biopsy and is performed prior to beginning the study and at the end of the first cycle, with additional bone marrow aspiration/biopsy performed every four weeks or at the end of subsequent cycles. Individuals also undergo biopsy to assess changes in progenitor cancer cell phenotype and clonogenic growth by flow cytometry, and for changes in cytogenetics by FISH or detection of chromosomal translocations by TaqMan PCR as a means to measure tumor burden. After completion of study treatment, individuals are followed periodically for 4 weeks.

An alternative approach includes testing AML individuals that have no real therapeutic options that have demonstrated efficacy. On humane ground, the request for an expedited Phase I/II combined trial is optionally proposed. In this instance, the initial dose escalation studies necessary to determine MTD in a Phase I is also accompanied by monitoring for clinical outcomes, rather than having to wait for Phase II.

### Example 10

Human Clinical Trial of the Safety and/or Efficacy of 5-(anthracen-1-ylmethylidene)-2-sulfanylidene-1,3diazinane-4,6-dione (or a pharmaceutically acceptable salt thereof) therapy

Objective: To determine the safety and pharmacokinetics of administered 5-(anthracen-1-ylmethylidene)-2-sulfanylidene-1,3diazinane-4,6-dione.

Study Design: This will be a Phase I, single-center, open-label, randomized dose escalation study followed by a Phase II study in leukemias individuals. Individuals should not have had exposure to 5-(anthracen-1-ylmethylidene)-2-sulfanylidene-1,3diazinane-4,6-dione prior to the study entry. Individuals must not have received treatment for their cancer within 2 weeks of beginning the trial. Treatments include the use of chemotherapy, hematopoietic growth factors, and biologic therapy such as monoclonal antibodies. The exception is the use of hydroxyurea for individuals with WBC>30×103/μL. This duration of time appears adequate for wash out due to the relatively short-acting nature of most anti-leukemia agents. Individuals must have recovered from all toxicities (to grade 0 or 1) associated with previous treatment. All subjects are evaluated for safety and all blood collections for pharmacokinetic analysis are collected as scheduled. All studies are performed with institutional ethics committee approval and individual consent.

Phase I: Individuals receive intravenous 5-(anthracen-1-ylmethylidene)-2-sulfanylidene-1,3diazinane-4,6-dione daily for 5 consecutive days or 7 days a week. Doses of 5-(anthracen-1-ylmethylidene)-2-sulfanylidene-1,3diazinane-4,6-dione may be held or modified for toxicity based on assessments as outlined below. Treatment repeats every 28 days in the absence of unacceptable toxicity. Cohorts of 3-6 individuals receive escalating doses of 5-(anthracen-1-ylmethylidene)-2-sulfanylidene-1,3diazinane-4,6-dione until the maximum tolerated dose (MTD) for the 5-(anthracen-1-ylmethylidene)-2-sulfanylidene-1,3diazinane-4,6-dione is determined. The MTD is defined as the dose preceding that at which 2 of 3 or 2 of 6 individuals experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Individuals receive 5-(anthracen-1-ylmethylidene)-2-sulfanylidene-1,3diazinane-4,6-dione as in phase I at the MTD determined in phase I. Treatment repeats every 6 weeks for 2-6 courses in the absence of disease progression or unacceptable toxicity. After completion of 2 courses of study therapy, individuals who achieve a complete or partial response may receive an additional 4 courses. Individuals who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 5-(anthracen-1-ylmethylidene)-2-sulfanylidene-1,3diazinane-4,6-dione. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Individuals undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Individual Response to therapy: Individual response is assessed with bone marrow aspiration/biopsy and is performed prior to beginning the study and at the end of the first cycle, with additional bone marrow aspiration/biopsy performed every four weeks or at the end of subsequent cycles. Individuals also undergo biopsy to assess changes in progenitor cancer cell phenotype and clonogenic growth by flow cytometry, and for changes in cytogenetics by FISH or detection of chromosomal translocations by TaqMan PCR as a means to measure tumor burden. After completion of study treatment, individuals are followed periodically for 4 weeks.

An alternative approach includes testing AML individuals that have no real therapeutic options that have demonstrated efficacy. On humane ground, the request for an expedited Phase I/II combined trial is optionally proposed. In this instance, the initial dose escalation studies necessary to determine MTD in a Phase I is also accompanied by monitoring for clinical outcomes, rather than having to wait for Phase II.

### Example 11

Human Clinical Trial of the Safety and/or Efficacy of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide (or a pharmaceutically acceptable salt thereof) therapy

Objective: To determine the safety and pharmacokinetics of administered 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide.

Study Design: This will be a Phase I, single-center, open-label, randomized dose escalation study followed by a Phase II study in cancer individuals with a cancer that can be biopsied (e.g., lymphoma). Individuals should not have had exposure to 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide prior to the study entry. Individuals must not have received treatment for their cancer within 2 weeks of beginning the trial. Treatments include the use of chemotherapy, hematopoietic growth factors, and biologic therapy such as monoclonal antibodies. The exception is the use of hydroxyurea for individuals with WBC>30×103/μL. This duration of time appears adequate for wash out due to the relatively short-acting nature of most anti-leukemia agents. Individuals must have recovered from all toxicities (to grade 0 or 1) associated with previous treatment. All subjects are evaluated for safety and all blood collections for pharmacokinetic analysis are collected as scheduled. All studies are performed with institutional ethics committee approval and individual consent.

Phase I: Individuals receive intravenous 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide daily for 5 consecutive days or 7 days a week. Doses of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide may be held or modified for toxicity based on assessments as outlined below. Treatment repeats every 28 days in the absence of unacceptable toxicity. Cohorts of 3-6 individuals receive escalating doses of 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide until the maximum tolerated dose (MTD) for the 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide is determined. The MTD is defined as the dose preceding that at which 2 of 3 or 2 of 6 individuals experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Individuals receive 4-amino-7-[(2R,3S,4S,5R)-3,4-dihydroxy-5-(hydroxymethyl)oxolan-2-yl]-6-hydrazinylpyrrolo[5,4-d]pyrimidine-5-carboxamide as in phase I at the MTD determined in phase I. Treatment repeats every 6 weeks for 2-6 courses in the absence of disease progression or unacceptable toxicity. After completion of 2 courses of study therapy, individuals who achieve a complete or partial response may receive an additional 4 courses. Individuals who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 4-amino-7-[(2R,3 S,4 S,5R)-3,4-dihydroxy-5-(hydroxymethyl) oxolan-2-yl]-6-hydrazinylpyrrolo[5,4-d]pyrimidine-5-carboxamide. Venous blood samples (5 mL) for determination of scrum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Individuals undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Individual Response to therapy: Individual response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each individual's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Individuals also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, individuals are followed periodically for 4 weeks.

### Example 12

Human Clinical Trial of the Safety and/or Efficacy of 2-[(2-chloro-4-nitrophenanthridin-6-yl)amino]ethanol (or a pharmaceutically acceptable salt thereof) therapy

Objective: To determine the safety and pharmacokinetics of administered 2-[(2-chloro-4-nitrophenanthridin-6-yl)amino]ethanol.

Study Design: This will be a Phase I, single-center, open-label, randomized dose escalation study followed by a Phase II study in cancer individuals with a cancer that can be biopsied (e.g., lymphoma). Individuals should not have had exposure to 2-[(2-chloro-4-nitrophenanthridin-6-yl)amino]ethanol prior to the study entry. Individuals must not have received treatment for their cancer within 2 weeks of beginning the trial. Treatments include the use of chemotherapy, hematopoietic growth factors, and biologic therapy such as monoclonal antibodies. The exception is the use of hydroxyurea for individuals with WBC>30×103/μL. This duration of time appears adequate for wash out due to the relatively short-acting nature of most anti-leukemia agents. Individuals must have recovered from all toxicities (to grade 0 or 1) associated with previous treatment.

All subjects are evaluated for safety and all blood collections for pharmacokinetic analysis are collected as scheduled. All studies are performed with institutional ethics committee approval and individual consent.

Phase I: Individuals receive intravenous 2-[(2-chloro-4-nitrophenanthridin-6-yl)amino]ethanol daily for 5 consecutive days or 7 days a week. Doses of 2-[(2-chloro-4-nitrophenanthridin-6-yl)amino]ethanol may be held or modified for toxicity based on assessments as outlined below. Treatment repeats every 28 days in the absence of unacceptable toxicity. Cohorts of 3-6 individuals receive escalating doses of 2-[(2-chloro-4-nitrophenanthridin-6-yl)amino]ethanol until the maximum tolerated dose (MTD) for the 2-[(2-chloro-4-nitrophenanthridin-6-yl)amino]ethanol is determined. The MTD is defined as the dose preceding that at which 2 of 3 or 2 of 6 individuals experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Individuals receive 2-[(2-chloro-4-nitrophenanthridin-6-yl)amino]ethanol as in phase I at the MTD determined in phase 1. Treatment repeats every 6 weeks for 2-6 courses in the absence of disease progression or unacceptable toxicity. After completion of 2 courses of study therapy, individuals who achieve a complete or partial response may receive an additional 4 courses. Individuals who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 2-[(2-chloro-4-nitrophenanthridin-6-yl)amino]ethanol. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Individuals undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak scrum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Individual Response to therapy: Individual response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each individual's study course. Response rates are determined using the RECIST criteria. (Therasse et al, J. Natl. Cancer Inst. 2000 Feb. 2; 92(3):205-16; http://ctep.cancer.gov/forms/TherasseRECTSTJNCI.pdf). Individuals also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. Aftcr completion of study treatment, individuals are followed periodically for 4 weeks.

### Example 13

Human Clinical Trial of the Safety and/or Efficacy of 5-(anthracen-1-ylmethylidene)-2-sulfanylidene-1,3diazinane-4,6-dione (or a pharmaceutically acceptable salt thereof) therapy

Objective: To determine the safety and pharmacokinetics of administered 5-(anthracen-1-ylmethylidene)-2-sulfanylidene-1,3diazinane-4,6-dione.

Study Design: This will be a Phase I, single-center, open-label, randomized dose escalation study followed by a Phase 11 study in cancer individuals with a cancer that can be biopsied (e.g., lymphoma).

Individuals should not have had exposure to 5-(anthracen-1-ylmethylidene)-2-sulfanylidene-1,3-diazinane-4,6-dione prior to the study entry. Individuals must not have received treatment for their cancer within 2 weeks of beginning the trial. Treatments include the use of chemotherapy, hematopoietic growth factors, and biologic therapy such as monoclonal antibodies. The exception is the use of hydroxyurea for individuals with WBC>30×103/μL. This duration of time appears adequate for wash out due to the relatively short-acting nature of most anti-leukemia agents. Individuals must have recovered from all toxicities (to grade 0 or 1) associated with previous treatment. All subjects are evaluated for safety and all blood collections for pharmacokinetic analysis are collected as scheduled. All studies are performed with institutional ethics committee approval and individual consent.

Phase I: Individuals receive intravenous 5-(anthracen-1-ylmethylidene)-2-sulfanylidene-1,3diazinane-4,6-dione daily for 5 consecutive days or 7 days a week. Doses of 5-(anthracen-1-ylmethylidene)-2-sulfanylidene-1,3diazinane-4,6-dione may be held or modified for toxicity based on assessments as outlined below. Treatment repeats every 28 days in the absence of unacceptable toxicity. Cohorts of 3-6 individuals receive escalating doses of 5-(anthracen-1-ylmethylidene)-2-sulfanylidene-1,3diazinane-4,6-dione until the maximum tolerated dose (MTD) for the 5-(anthracen-1-ylmethylidene)-2-sulfanylidene-1,3diazinane-4,6-dione is determined. The MTD is defined as the dose preceding that at which 2 of 3 or 2 of 6 individuals experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Individuals receive 5-(anthracen-1-ylmethylidene)-2-sulfanylidene-1,3diazinane-4,6-dione as in phase I at the MTD determined in phase I. Treatment repeals every 6 weeks for 2-6 courses in the absence of disease progression or unacceptable toxicity. After completion of 2 courses of study therapy, individuals who achieve a complete or partial response may receive an additional 4 courses. Individuals who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 5-(anthracen-1-ylmethylidene)-2-sulfanylidene-1,3diazinane-4,6-dione. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Individuals undergo plasma/scrum sample collection for pharmacokinetic evaluation before beginning treatment and at days 1, 2, 3, 4, 5, 6, 7, and 14. Pharmcokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Individual Response to therapy: Individual response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each individual's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Individuals also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, individuals are followed periodically for 4 weeks.

### Example 14

### Parenteral Composition

An i.v. solution is prepared in a sterile isotonic solution of water for injection and sodium chloride (˜300 mOsm) at pH 11.2 with a buffer capacity of 0.006 mol/l/pH unit. The protocol for preparation of 100 ml of a 5 mg/ml a first and/or second agent for i.v. infusion is as follows: add 25 ml of NaOH (0.25 N) to 0.5 g of the active agent and stir until dissolved without heating. Add 25 ml of water for injection and 0.55 g of NaCl and stir until dissolved. Add 0.1N HCl slowly until the pH of the solution is 11.2. The volume is adjusted to 100 mL. The pH is checked and maintained between 11.0 and 11.2. The solution is subsequently sterilized by filtration through a cellulose acetate (0.22 μm) filter before administration.

### Example 15

### Oral Composition

A pharmaceutical composition for oral delivery is prepared by mixing 100 mg of the active with 750 mg of a starch. The mixture is incorporated into an oral dosage unit, such as a hard geletin capsule or coated tablet, which is suitable for oral administration.

It should be understood that various alternatives to the embodiments described herein may be employed in practicing the invention. It is intended that the following claims define the scope of the invention and that methods and structures within the scope of these claims and their equivalents be covered thereby.

1-93. (canceled)

94. A process for identifying a therapeutic agent that selectively inhibits the growth of, or induces apoptosis in, in a cancer stem cell by:
a. identifying at least one candidate compound;
b. contacting a plurality of normal conditionally immortalized hematopoietic stem cells with the candidate compound;
c. contacting a plurality of leukemic cancer stem cells with the candidate compound;
d. detecting or measuring the impact of the candidate compound on the viability of the plurality of normal conditionally immortalized hematopoietic stem cells and the plurality of leukemic cancer stem cells; and
e. comparing the impact of the candidate compound on viability of the plurality of normal conditionally immortalized hematopoietic stem cells to the impact of the candidate compound on viability of the plurality of leukemic cancer stem cells.

95. The process of claim 94, wherein the plurality of normal conditionally immortalized hematopoietic stem cells is generated by a method comprising contacting a population of hematopoietic stem cells with recombinant MYC-ER and BCL-2 polypeptides.

96. The process of claim 94, wherein the plurality of normal conditionally immortalized hematopoietic stem cells is generated by a method comprising contacting a population of hematopoietic stem cells with: a recombinant MYC-ER polypeptide, wherein the MYC-ER polypeptide is selected from Tat-MYC-ER or Vpr-MYC ER; and a recombinant BCL-2 polypeptide, wherein the BCL-2 polypeptide is Tat-Bcl-2 or Vpr-Bcl-2.

97. The process of claim 94, wherein the plurality of leukemic cancer stem cell is generated by a method comprising:
a. contacting a population of hematopoietic stem cells with (1) recombinant MYC-ER polypeptide, wherein the MYC-ER polypeptide is selected from Tat-MYC-ER or Vpr-MYC ER; and (ii) recombinant BCL-2 polypeptide, wherein the BCL-2 polypeptide is Tat-Bcl-2 or Vpr-Bcl-2; and
b. limiting dilution of the population of hematopoietic stem cells without any helper or feeder cells.

98. The process of claim 94, wherein detecting or measuring the impact of the candidate compound on viability of the normal conditionally immortalized hematopoietic stem cell and the leukemic cancer stem cell is achieved by 7AAD staining, a GFP viability assay, or a combination thereof.

99. A therapeutic agent identified by the process of claim 94.

100. A method of selectively inducing apoptosis in or inhibiting the growth of a cancer stem cell relative to a normal hematopoietic stem cell, comprising contacting the cell with an effective amount of a compound identified by the process of claim 94.

101. The method of claim 100, wherein the cancer stem-cell is a hematological cancer stem cell.

102. The method of claim 100, wherein the cancer stem cell is a leukemic stem cell.

103. The method of claim 100, wherein the cancer stem cell is present in an individual diagnosed with, is suspected of having, or is predisposed to develop cancer.
